(19) United States
(12) Patent Application Publication
(10) Pub. No.: US 2004/0258664 A1
(43) Pub. Date: Dec. 23, 2004

(54) Multifunctional complex for targeting specific phagocytosis of a target agent

(76) Inventors: Jacob Pitcovski, Korazim (IL); Ely Morag, Kfar Aviv (IL); Yosef Pinchasov, Beit Gamliel (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/488,402

(22) PCT Filed: Mar. 10, 2003
(86) PCT No.: PCT/IL03/00196

(30) Foreign Application Priority Data
Mar. 10, 2002 (IL) 148598

(51) Int. Cl. A61K 48/00; A61K 39/40; A61K 39/42

(52) U.S. Cl. 424/93.2; 424/178.1

(57) ABSTRACT

AbstractThe present invention relates to a multi-functional targeting complex for inducing a specific immuno-stimulatory reaction, preferably, phagocytosis, against at least one target pathogenic agent. The complex of the invention comprises at least one target recognition component comprising a molecule that specifically binds to the desired target agent, an immuno-active component comprising an immuno-stimulatory agent; and optionally, a connecting component that associates the targeting component and the immuno-active component. The complex of the invention provides an effective therapeutic prevention and treatment of various pathogenic disorders, such as mastitis in caws and furunculosis in fish. The invention further relates to compositions comprising the targeting complex, methods of treatment and uses thereof.

### FIELD OF THE INVENTION

The present invention relates to a targeting complex comprising a targeting component and an immuno-stimulatory active component associated together, optionally via a connecting component, which complex provides an effective therapeutic prevention and treatment of various pathogenic disorders.

### BACKGROUND OF THE INVENTION

Vaccines are the most efficient way to prevent diseases caused by pathogens like viruses and bacteria. A vaccine protects the animal by stimulating specific cells and antibodies of the immune system. In active vaccination, humans or animals are exposed to an attenuated, inactivated or toxin form of the disease agent. In response, antibodies and memory cells are produced that confer protection for short or long terms, respectively. In absence of an active vaccine, an unprotected animal may become infected and needs to be treated by various medicaments, e.g. antibiotics in the case of bacteria. One of the solutions is passive vaccination. In this approach, antibodies against a specific pathogen, produced in one animal, are administered to another animal which is infected by the same pathogen. One example is maternal immunity. For example, E. coli K99, infects young calves and causes diarrhea. Infected calves may be protected by maternal antibodies directed against the bacteria, which are supplied via the colostrum. Hens passively transfer antibodies to their offsprings, the developing chicks, through egg yolk or through the blood [Hanson et al., Science and Medicine 4:12-21 (1997)], while transfer of antibodies in mammal is via the colostrum.

Numerous publications [Brunser, et. al, 1992; Campbell and Petersen, Immune Milk. Journal of Immune Milk 1:3-28 (1964); Davidson, et. al, The Lancet 2:709-712 (1989); Ebina, et. al, Medical Microbiology and Immunology 174:177-185 (1985); Fayer, W. et. al., 1989, Golay, et. al., 1990; Mietens and Keinhorst, 1979; Ormrod and Miller, 1991, 1993, Owens and Nickerson, 1988, Sharpe, et. al, 1994; Smith, C. M. (1964)] report that multiple inoculations of hens or dairy cows over a long period of time with an antigen (i.e., hyperimmunization) results in the production of “immune” eggs or milk. These products have antibodies against the specific vaccinating antigens.

Similarly to in utero transferred immune protection is observed in mammals, hens passively transfer protection to their progeny by secreting antibodies into the egg. Transfer of chicken antibodies from the hen's serum to the yolk, and absorption by the chick, is analogous to cross-placental transfer of IgG from the mammalian mother to its offspring [Kowalezyk, et al., Transport in the Chicken Immunology 54:755-762 (1985); Rose, et al., European Journal of Immunology 4:521-523 (1974)].

Since immunoglobulins occur naturally in eggs, and egg products are a common source of protein in human diet, immune eggs are a convenient and economical source of specific antibodies [Hamada, et al., Infection and Immunity 59:4161-4167 (1991); Polson, et al., Immunological Investigations 14:323-327 (1985)]. The immune egg therefore appears to serve as a concentrated source of antibodies.

It is therefore one of the objects of the present invention to use antibodies raised in eggs (IgY), as a targeting component in the targeting complex of the invention. These targeting components (IgY) specifically direct immunostimulatory response against a particular pathologic agent.

The immunoactive component of the targeting complex of the present invention comprises a phagocytosis inducing agent such as complement, bacterial antibodies or IgG which induces opsonization by its Fc fragment. Both components, the targeting component and the immunoactive component of the targeting complex of the present invention, may be preferably bound to a micro/nano carrier.

Immunostimulatory cell surface polypeptides and their receptors are important for the clearance and destruction of foreign materials, including mammalian cells or bacteria. Immunostimulatory cell surface polypeptides and their receptors activate the phagocytosis and ADCC. The process begins with opsonization of the foreign materials. An opsonin is an agent, usually an antibody or complement components, that makes a cell or microbe more vulnerable to being engulfed by a phagocyte. Opsonization is the process of coating a cell with opsonin. A phagocyte is a cell that engulfs and devours another, the process of engulfing and devouring is phagocytosis. Amongst the important phagocytes for the purpose of this invention are leukocytes such as macrophages and monocytes.

The present invention thus provides for a targeted immuno-stimulatory complex, for the prevention and treatment of variety of pathological disorders caused by pathogenic agents (bacteria, virus), toxins and other antibiotic-resistant pathogens, as well as malignant disorders in a mammalian subject in need.

In a particular example for targeted immunostimulation, the present invention further provides targeted complex pharmaceutical compositions and method for the prevention and/or treatment of bovine mastitis. The targeted complex comprises the active immunostimulatory component and a targeting component, which is an IgY antibody specific for a mastitis pathogen. Both components may be preferably attached to a micro/nano carrier bead.

Bovine mastitis involves inflammatory changes of the lactiferous ducts, and generally refers to inflammation of the duct system or mammary tissue as a result of the colonization by, or proliferation of, microbes invading the ducts or teat cistern. The milk synthesizing function is thus compromised, and the over-permeation of the cell walls secreting the milk results in the secretion of abnormal milk, with an increase in the number of somatic cells, particularly white blood cells. Additionally, lactating cell disorders and atrophy, as well as the proliferation of connective tissue and the like can also lead to reduced or absent milk production.

The major mastitis pathogens include Staphylococcus, Streptococcus, Actinomyces, Escherichia coli, Pseudomonas, Clostridium and fungi.

The mechanism of mastitis infection or onset is complex, and involves (in terms of bacteria) bacterial infiltration of pathogens, their colonization on the mammary epithelia, and their subsequent proliferation or tissue invasion. Predisposing factors may include genetics, constitution, climate, livestock facilities, environment, the number of feeding animals, feed, milking methods, papillary trauma, and the like, but for practical purposes, the most important is milking hygiene, including proper handling by milkers.

Practical damage caused by mastitis includes lower amounts of milk, lower milk quality, milk waste, the absence of milk, reduced number of years of animal use, greater treatment costs, and potential sources for the infection of healthy cows, all of which represent profound economic losses. Mastitis has thus been the subject of considerable research in many countries over the past 200 years. Although most results have been put to use in the treatment and prevention of the condition itself, its incidence has not declined, and has even increased in some areas. Since mastitis involves inflammatory changes of the mammary glands resulting from microbial infection, responses generally include the intra-mammary administration of antibiotics and synthetic anti-bacterials. Administration of antibiotic preparations and synthetic antibacterials is followed by a period in which the milk cannot be used, in concern for the health of human consumers. Since the milk produced during that period is of no commercial value, it represents a major financial loss. In addition, the dosage and administration of the drugs must, of course, be supervised in the area where the treatment is undertaken, but drugs are sometimes selected without testing the susceptibility of the pathogens thereto. Many pathogens have thus acquired multiple resistances, often preventing the establishment of an effective treatment plan.

It is therefore another object of the present invention to overcome the aforementioned problems in providing a simple yet practical and economical method and targeting complex for preventing and treating mastitis without leaving any drugs in milk or producing drug-resistant bacterial strains.

These and other objects of the invention will become apparent as the description precedes.

### SUMMARY OF THE INVENTION

In a first aspect, the present invention relates to a multi-functional targeting complex that induces a specific immuno-stimulatory reaction to at least one target agent. The targeting complex of the invention comprises:

(a) at least one target recognition component, comprising a molecule which specifically binds to said agent or to any functional fragment of said agent;

(b) an active component comprising an immuno-stimulatory agent; and

(c) optionally, a connecting component that associates at least one of said targeting component and at least one of said active component. The association of said targeting component and said active component, optionally by said connecting component, should be performed in the appropriate conformation in order to the retain the independent biological functions of both the active and the targeting components.

In one embodiment, the targeting complex of the invention induces a specific immuno-stimulatory reaction selected from the group consisting of phagocytosis, antibody-dependent cell-mediated cytotoxicity (AD CC), complement activation, and stimulation of specific T cells and B cells. This specific immuno-stimulatory reaction is targeted by the complex of the invention to at least one target pathologic agent.

In a specifically preferred embodiment, the targeting complex of the invention induces a specific phagocytosis by leukocytes such as any one of macrophages and monocytes, targeted to at least one target agent.

According to another embodiment, the targeting complex of the invention is targeted against a target pathologic agent selected from the group consisting of bacteria, viruses, fungi, malignant cells, toxins, venoms, haptens and undesired proteins. More preferably, the target pathologic agent may be bacteria selected from the group consisting of mastitis pathogenic bacteria, enteropathogenic bacteria and pathogenic furonculosis bacteria. In a most specifically preferred embodiment, the targeting complex of the invention is targeted to at least one mastitis pathogenic bacteria selected from the group consisting of Actinomyces, E. coli, Pseudomonas, Clostridium, Staphylococcus aureus, Streptococcus agalactiae, Streptococcus dysgalactia, Corynebacterium pyogenes, Enterococcus faecalis, Corynebacterium bovis, Nocardia asteroides (or farcinica) and Streptococcus uberis.

Alternatively, the target recognition component of the targeting complex may be targeted against Aeromonas spp., particularly, Aeromonas salmonicida, which causes an ulcerative disease in fish.

In yet another particular embodiment, the target recognition component comprised within the targeting complex of the invention, comprises any one of an antibody or any functional fragments thereof, ligands and receptors specific to said pathologic agent or to any functional fragment of said agent. In a specifically preferred embodiment, the target recognition component comprises an antibody or any functional fragments thereof. Such antibody is specific to said pathologic agent or to any functional fragment of said agent, and may be for example any one of IgG, IgY and IgM, preferably, an IgY.

According to a specific embodiment, the targeting complex of the invention comprises as the active component an immuno-stimulatory agent. More particularly, this immuno-stimulatory agent induces phagocytosis. The phagocytosis inducing agent comprised within the targeting complex of the invention as an active component, may be selected from the group consisting of opsonins, which are any one of, but not limited to, IgG and C3b, protein molecules having carbohydrate residues that interact with the mannose-fucose receptor of phagocytes, ligand protein molecules recognized by receptors on scavenger macrophages, ligands for integrins expressed by phagocytes; glycoproteins and fucosyl transferase.

According to a specifically preferred embodiment, the phagocytosis inducing agent is IgG.

According to anther preferred embodiment, the optional connecting component comprised within the targeting complex of the invention, may be any one of a solid support, a chemical linker, an amino acid residue, a peptide, oligopeptide or polypeptide linker, a saccharide, oligosaccharide or polysaccharide linker and a lipid linker.

In yet another embodiment, the complex of the invention comprises a targeting component and an immunoactive component, both attached without any connecting component. According to such embodiment, the targeting component may be an IgY antibody directed against said target agent, and the immunoactive component is an IgG anti IgY antibody. Binding of both component retains the appropriate conformation (having the Fc of the IgG molecule free).

In a particular embodiment, the invention relates to a multi-functional targeting complex that induces specific phagocytosis of at least one target mastitis pathogenic bacteria in bovine infected by said bacteria. This specific complex comprises:

a. at least one target recognition component comprising a molecule which specifically binds to the mastitis pathogenic bacteria or to any functional fragment of said bacteria;

b. at least one active component comprising a phagocytosis-inducing agent; and

c. optionally, a connecting component that associates at least one of said targeting component and at least one of said active component. Both active and targeting components are associated, optionally by the connecting component, in an appropriate conformation that retains the independent biological functions of both components.

This targeting complex according to a specific embodiment comprises as a target recognition component, an IgY antibody specific for mastitis pathogenic bacteria. More specifically, the targeting complex of the invention may comprise at least one IgY antibody specific for at least one of the mastitis pathogenic bacteria selected from the group consisting of Actinomyces, E. coli, Pseudomonas, Clostridium, Staphylococcus aureus, Streptococcus agalactiae, Streptococcus dysgalactiae, Corynebacterium pyogenes, Enterococcus faecalis, Corynebacterium bovis, Nocardia asteroides (or farcinica) and Streptococcus uberis.

According to a specifically preferred embodiment, the targeting complex of the invention comprises as a phagocytosis-inducing agent, an IgG molecule. Most preferably, said IgG molecule is generated in bovine.

This particular targeting complex may optionally further comprise as a connecting component, any one of, but not limited to, a solid support, a chemical linker, a peptide, oligopeptide or polypeptide linker, a saccharide, oligosaccharide or polysaccharide linker and lipid linker.

The invention further provides for a multi-functional targeting complex for inducing specific phagocytosis of at least one target pathogenic bacteria causing ulcerative disease in fish infected by said bacteria. According to this embodiment, such specific complex comprises: (a) at least one target recognition component comprising a molecule that specifically binds to Aeromonas spp. pathogenic bacteria which causes ulcerative disease in fish, or to any functional fragment of said bacteria; (b). an active component comprising phagocytosis inducing agent; and (c) optionally, a connecting component that associates at least one of said targeting component and at least one of said active component, wherein the independent biological functions of said targeting component and said active component are retained.

As a further aspect, the invention relates to a composition for the treatment of a pathologic disorder in a subject. The composition of the invention comprises as active ingredient a multi-functional targeting complex that induces a specific immuno-stimulatory reaction to at least one target pathologic agent. The complex comprised within the composition of the invention comprises: (a) at least one target recognition component comprising a molecule which specifically binds to said pathologic agent or to any functional fragment of said agent; (b) at least one active component comprising an immuno-stimulatory agent; and (c) optionally, a connecting component that associates said at least one targeting component and said at least one active component.

The composition of the invention may optionally further comprise pharmaceutically and/or veterinarily acceptable diluent, excipient and/or additive.

According to one embodiment, the composition of the invention is intended for the treatment of a pathological disorder that may be any one of immune-related disorders, viral, fungal or bacterial infections and malignant disorders, in a subject in need of such treatment.

Such subject may be according to a preferred embodiment, any one of human, domestic mammal, domestic bird, domestic aquaculture and exotic aquaculture.

In one preferred embodiment, the composition of the invention is intended for the treatment of domestic mammals, most preferably, bovines, and particularly dairy cows.

In a particular embodiment, the pathologic disorder to be treated with the composition of the invention may be a bacterial infection. More preferably, the bacterial infection may be bovine mastitis.

Therefore, the invention further provides a composition for the treatment of mastitis in bovines. Such specific composition according to one embodiment comprises as active ingredient a multi-functional targeting complex, that induces specific phagocytosis to at least one target mastitis pathogenic bacteria in a bovine infected by said bacteria. More specifically, the complex comprised within this specific composition for mastitis, comprises: (a) at least one target recognition component comprising a molecule which specifically binds to said mastitis pathogenic bacteria or any functional fragment of said bacteria; (b) an active component comprising phagocytosis inducing agent; and (c) optionally, a connecting component that associates at least one of said targeting component and said active component.

According to a specifically preferred embodiment the composition of the invention comprises as an active ingredient, a complex comprising an avidin-coated polystyrene bead as a connecting component, a biotinylated IgY antibody specific for mastitis pathogenic bacteria, as the targeting component, and an anti avidin IgG molecule, which is, most preferably, generated in bovine.

In another embodiment, the composition of the invention is intended for the treatment of domestic fish and pet/aquarium/exotic fish, which suffers of ulcerative disease caused by bacterial infection of Aeromonas spp. According to this embodiment, the invention therefore provides for a composition for the treatment of ulcerative disease in fish, comprising as active ingredient a multi-functional targeting complex, that induces specific phagocytosis of at least one target Aeromonas spp. pathogenic bacteria in fish infected by said bacteria. Such particular complex comprises: (a) at least one target recognition component comprising a molecule which specifically binds to said Aeromonas spp. pathogenic bacteria or any functional fragment of said bacteria, preferably an IgY antibody specific for Aeromonas spp.; (b) an active component comprising phagocytosis inducing agent, which may preferably be an IgG molecule prepared in fish; and (c) optionally, a connecting component that associates at least one of said targeting component and at least one of said active component, wherein the independent biological functions of said targeting component and said active component are retained.

The present invention further relates to a composition for inducing a specific phagocytosis targeted to at least one target pathologic agent or compound related to a pathological disorder in a subject suffering from said disorder. According to one embodiment, this composition comprises as an active ingredient, the multi-functional targeting complex of the invention, and optionally further comprises pharmaceutically and/or veterinarily acceptable diluent, excipient and/or additive.

According to a specific embodiment the invention relates to a composition for inducing a specific phagocytosis of at least one target mastitis bacterial pathogen in a bovine suffering from mastitis. This composition comprises as an active ingredient, the specific complex according to the invention and optionally further comprises pharmaceutically and/or veterinarily acceptable diluent, excipient and/or additive.

Another specific embodiment relates to a composition for inducing a specific phagocytosis of at least one target Aeromonas spp. bacterial pathogen in a fish suffering from ulcerative disease, which composition comprises as an active ingredient a multi-functional targeting complex according to the invention, and optionally further comprises pharmaceutically and/or veterinarily acceptable diluent, excipient and/or additive

As a third aspect, the present invention relates to a method for the treatment of a pathological disorder in a subject. The method of the invention comprises administering to the subject a therapeutically effective amount of a targeting complex or a pharmaceutical composition comprising the same. This complex comprises: (a) at least one target recognition component comprising a molecule which specifically binds to said pathologic agent or to any functional fragment of said agent; (b) at least one active component comprising an immuno-stimulatory agent; and (c) optionally, a connecting component that associates at least one of said targeting component and at least one of said active component.

The association of said targeting component and said active component, optionally by said connecting component, should be performed in the appropriate conformation in order to the retain the independent biological functions of both the active and the targeting components.

In one embodiment, the method of the invention utilizes any one of the compositions according to the invention. More preferably, these compositions comprise as an effective ingredient any one of the targeting complexes of the invention.

In yet another embodiment, the method of the invention is intended for the treatment of a pathological disorder which may be any one of immune-related disorders, viral, fungal or bacterial infections and malignant disorders. In a specifically preferred embodiment, the pathological disorder may be a bacterial infection.

In a particularly preferred embodiment, the invention relates to a method for the treatment of mastitis in cattle. This specific method comprises administering to the animal in need a therapeutically effective amount of the targeting complex capable of inducing specific phagocytosis targeted to at least one target mastitis pathogenic bacteria in said infected animal, or of a pharmaceutical composition comprising the same.

In yet another particularly preferred embodiment, the method of the invention utilizes a particular composition of the invention. According to this embodiment, this composition comprises as an active ingredient, a complex comprising an avidin coated polystyrene bead as a connecting component, a biotinylated IgY antibody specific for mastitis pathogenic bacteria, as the targeting component, and an anti-avidin IgG molecule, which is, most preferably, generated in bovine. This particular composition may optionally further comprise pharmaceutically and/or veterinarily acceptable diluent, excipient and/or additive.

The present invention further provides for a method for the treatment of ulcerative disease in fish comprising administering to said fish in need a therapeutically effective amount of a targeting complex capable of inducing specific phagocytosis targeted to at least one target Aeromonas spp. pathogenic bacteria in said infected fish according to the invention, or of a pharmaceutical composition comprising the same.

The present invention further relates to the use of a multi-functional targeting complex of the invention, in the preparation of a pharmaceutical composition for the treatment of a pathological disorder in a subject in need.

This pathological disorder may be according to a specific embodiment any one of immune-related disorders, viral, fungal or bacterial infections and malignant disorders. Preferably, the pathologic disorder is a bacterial infection.

In another specifically preferred embodiment, the invention relates to the use of a specific multi-functional targeting complex, in the preparation of a pharmaceutical composition for the treatment of mastitis in cattle. This particular complex comprises an avidin-coated polystyrene bead as a connecting component, a biotinylated IgY antibody specific for mastitis pathogenic bacteria, as the targeting component, and an anti-avidin IgG molecule, which is most preferably, generated in cattle.

Still further, the invention relates to the use the multi-functional targeting complex of the invention, in the preparation of a pharmaceutical composition for the treatment of ulcerative disease in fish.

### BRIEF DESCRIPTION OF THE FIGURES

FIG. 1. Antibody activity of crude egg yolk anti-E. Coli IgY soluble extract (1:1000) with time post-immunization determined by ELISA (O.D at 450 nm). Abbreviations: D. Po. Imm. (days post immunization), OD (optical density).

FIG. 2. Antibody activity of crude egg yolk anti-Staphylococcus aureus IgY soluble extract (1:100) with time post-immunization determined by ELISA (O.D at 450 nm). Abbreviations: D. Po. Imm. (days post immunization), OD (optical density).

FIG. 3. Antibody activity of crude egg yolk anti-Streptococcus uberis IgY soluble extract (1:1000) with time post-immunization determined by ELISA (O.D at 450 nm). Abbreviations: D. Po. Imm. (days post immunization), OD (optical density).

FIG. 4. The effect of anti-E. Coli IgY on the growth pattern of bovine E. Coli. Abbreviations: incu. T. min. (incubation time in minutes), cont. (control), a (anti), n. spec. (non-specific), IgY (immunoglobulines Y).

FIG. 5. The effect of anti-E. Coli IgY on the growth of bovine E. Coli. Abbreviations: incu. T. min. (incubation time in minutes), cont. (control), a (anti), n. spec. (non-specific), IgY (immunoglobulines Y).

FIG. 6. The targeting complex action:

Two major components are bound to the surface of the solid support (micro beads=mic. b.): (i) a targeting component (tar. comp.) comprising an antibody (IgY, receptors, IgG) specifically recognizing the target (pathogen or other target molecules) (e.g. Hormones, tumor specific antigens, viral proteins) and (ii) an active component having immuno-stimulating properties, which induces phagocytosis (IgG, complement). The targeting component binds to a specific target whereas the active component induces a non-specific host immune response (e.g. phagocytes). This targeted stimulation of phagocytosis enables specific clearance of an undesired target from the treated mammalian subject.

FIG. 7. SCC in milk of cows treated (left quarters) or untreated (right quarters) with two consecutive injections of the complex of the invention targeted to S. dysgalactia. Abbreviations: SCC(somatic cell counts), Trea. (treated), N-Trea. (non-treated), D. Po. treat. (days post treatment).

FIG. 8. The level of the S. dysgalactia specific complex in milk of three cows during 72 hours post injection. Abbreviations: Co. no. (cow number), T. Po. Infec. Hrs. (time post infection in hours).

FIG. 9. The level of S. dysgalactia specific complex in milk of three cows during first 6 hours after intra-mammary injection (Means±SD). The time point five hours, indicates pre-milking status and six hours indicates post-milking status.

### DETAILED DESCRIPTION OF THE INVENTION

A number of methods of the art of molecular biology are not detailed herein, as they are well known to the person of skill in the art. Such methods include expression of cDNAs, analysis of recombinant proteins or peptides, transformation of cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y.

As a first aspect, the present invention relates to a multi-functional targeting complex that induces a specific immuno-stimulatory reaction to at least one target agent, preferably a pathologic agent. The targeting complex of the invention comprises: (a) at least one target recognition component comprising a molecule which specifically binds to said agent or to any functional fragment of said agent; (b) at least one immunoactive component comprising an immuno-stimulatory agent; and (c) optionally, a connecting component that associates said at least one targeting component and said at least one active component, wherein the association of the targeting component and the active component, optionally via said connecting component, is such that appropriate conformation is preserved which so that the independent biological functions of both the active and the targeting components are preserved.

In one embodiment, the targeting complex of the invention induces a specific immuno-stimulatory reaction selected from the group consisting of phagocytosis, antibody-dependent cell-mediated cytotoxicity (AD CC), complement activation, and stimulation of specific T cells and B cells. This specific immuno-stimulatory reaction is targeted by the complex of the invention to at least one target pathologic agent.

As noted above, different components of the host immune system may be specifically induced by the targeting complex of the invention. In one component, phagocytes scavenge target pathologic agent, such as bacterial cells, malignant cells, undesired proteins or virus infected cells. In particular, antibody-dependent cell-mediated cytotoxicity (ADCC) has an important role in the destruction of many target cells, including tumor cells, by macrophages. Opsonization of target cells with immunoglobulin G (IgG) for example, enhances the removal of these materials from a host. The role of macrophages in the destruction of target cells by ADCC in the presence of specific antibodies has been well established. While the selectivity of macrophage targeting is based on antibody specificity, the lytic attack on the target cells is triggered by Fc receptor-mediated AD CC.

Another component of the immune system is the activation of the complement system. The two pathways of complement activation (the classical and the alternative pathways) are both directed at a central step in complement activation, the cleavage of C3. A single terminal pathway is the formation of a membrane attack complex (MAC). The classical pathway is normally activated by antigen-antibody complexes, where certain antibodies are complement fixing (capable of binding to complement to cause activation of the classical pathway). Activation of the classical pathway can be initiated with binding of C1q, the first factor of complement cascade, to the Fc region of immunoglobulin. Then, a cascade of proteolytic events results in the activation of C5 convertase, which cleaves C5 into C5b and C5a. The C5b then binds C6, C7, C8, to form a C5b-8 complex. Binding of C9 molecules to C5b-8 forms C5b-9 (the MAC), which penetrates into lipid bilayers and forms transmembrane channels that permit bidirectional flow of ions and macromolecules. By this mechanism, complement causes lysis of the target pathologic agent (such as cells, for example).

The targeting complex of the invention stimulates an immune response towards the target pathologic agent, since the activation of phagocytes, especially macrophages, acts to regulate both T and B lymphocytes. Macrophages engulf the target pathologic agent bound to the complex of the invention and present the antigenic determinants of the target agent to T cells, stimulating an immune response (Th1, and Th2 response), which results in activation of B cells and production of antibodies against said target agent (Th1 response).

In a specifically preferred embodiment, the targeting complex of the invention induces a specific phagocytosis by any leukocyte such as any one of macrophages and monocytes, targeted to at least one target agent. A phagocyte is a cell that engulfs and devours another; the process of engulfing and devouring is phagocytosis. Among the important phagocytes for this invention are macrophages and monocytes. Monocytes are a type of large white blood cells that travel in the blood but which can leave the bloodstream and enter tissue to differentiate into macrophages. Macrophages digest debris and foreign cells. Monocytes are generally characterized by the cell surface expression of CD 14.

According to another embodiment, the targeting complex of the invention is targeted against a target pathologic agent such as bacteria, viruses, fungi, malignant cells, toxins, venoms, haptens and undesired proteins. As non-limiting examples for bacterial targets the complex of the invention may be targeted to bacteria such as, but not limited to, Pseudomonas, Staphylococcus, Salmonella, enterotoxigenic E. coli, Salmonella typhimurium, Aeromonas spp., Anthrax and also to bacterial polysaccharide and most preferably to bacterial LPS (lipopolysaccaride). Viral targets may be for example, canine distemper virus, hepatitis in human, particularly, Hepatitis-B surface antigen, mouth and foot disease in cows, Newcastle disease virus in chicken and potato virus. Venoms may include rattlesnake and scorpion venom. The complex of the invention may also be targeted to different proteins such as transferrin, human plasmatic and platelet von Willebrand factor, to small bio-active peptides such as beta-Casokinin-10 or to haptens such as 1,25-dihydroxy-vitamin-D and also to environmental pollutants.

More preferably, the target pathologic agent may be bacteria selected from the group consisting of mastitis pathogenic bacteria, enteropathogenic bacteria and pathogenic furonculosis bacteria (Aeromonas salmonicida in fish).

In a most specifically preferred embodiment, the targeting complex of the invention is targeted to at least one mastitis pathogenic bacteria selected from the group consisting of Actinomyces, E. coli, Pseudomonas, Clostridium, Staphylococcus aureus, Streptococcus agalactiae, Streptococcus dysgalactiae, Corynebacterium pyogenes, Enterococcus faecalis, Corynebacterium bovis, Nocardia asteroides (or farcinica) and Streptococcus uberis.

In yet another specifically preferred embodiment, the targeting complex of the invention is targeted to at least one pathogenic bacteria, Aeromonas spp., which causes an ulcerative disease in fish, particularly exotic fish.

In yet another particular embodiment, the target recognition component comprised within the targeting complex of the invention, comprises any one of antibody or any functional fragments thereof, ligand and receptor specific to said pathologic agent or to any functional fragment of said agent.

In a specifically preferred embodiment, the target recognition component comprises an antibody or any functional fragments thereof. Such antibody is capable of specifically recognizing said pathologic agent or any functional fragment of said agent, and may be, for example, IgG, IgY or IgM. Preferably, the recognition component comprises an IgY antibody or any functional fragment thereof, specific to said pathologic agent or to any functional fragment of said agent.

The term “antibody” is meant to include both intact molecules and fragments thereof, such as, for example, Fab and F(ab′)₂, which are capable of binding antigen. Fab and F(ab′)₂ fragments lack the Fc fragment of intact antibody, clear more rapidly from the circulation, and may have less non-specific tissue binding than an intact antibody [Wahl et al., J. Nucl. Med. 24:316-325, (1983)]. It will be appreciated that Fab and F(ab′)₂ and other fragments of the antibodies, and particularly ScFv are useful in the present invention may be used as the targeting component of the complex, by the same methods disclosed herein for intact antibody molecules. Such fragments are typically produced by proteolytic cleavage, using enzymes such as papain (to produce Fab fragments) or pepsin (to produce F(ab′)₂ fragments) or by molecular procedures.

An antibody is said to be “capable of specifically recognizing” or “specific for” or “specific to” a certain target cell (bacterial cell or malignant or virus infected eukaryotic cell) if it is capable of specifically reacting with an antigen which is in this particular example an extracellular marker molecule expressed by said cell, to thereby bind the molecule to the antibody.

An “antigen” is a molecule or a portion of a molecule capable of being bound by an antibody, and which is additionally capable of inducing an animal to produce antibody capable of binding to an epitope of that antigen. An antigen may have one, or more than one epitope. The term “epitope” is meant to refer to that portion of any molecule capable of being bound by an antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

It is to be appreciated that functional fragments of the target, is any fragment that can be recognized by the targeting component of the complex of the invention. In a preferred embodiment when the targeting component is an antibody, the said fragment may be an epitope recognized by said targeting component.

According to a specific embodiment, the targeting complex of the invention comprises as the active component an immuno-stimulatory active component. More particularly, this immuno-stimulatory agent induces phagocytosis. Therefore, binding of the target component to the pathologic target agent, and activation of phagocytosis by said activating component, results in the selective phagocyte-mediated ablation of the undesirable target.

The phagocytosis-inducing agent comprised within the targeting complex of the invention as an immunoactive component, may be according to a specific embodiment, a polypeptide, naturally expressed on a cell surface, which is capable of stimulating an immune response against the cell in a host. Polypeptides appropriate for use as immunostimulatory cell surface polypeptides include, but are not limited to, the following:

a. opsonins such as IgG and C3b;

b. proteins with carbohydrate residues that interact with the mannose-fucose receptor of phagocytes;

c. proteins capable of recognition by receptors on scavenger macrophages;

d. ligands for integrins located on phagocytes;

e. glycoproteins, such as integrins and selectins; and

f. fucosyl transferase, which generates a Ga1-Ga1 epitope recognized by macrophages.

According to a specific embodiment, immunoglobulin G (IgG) is the preferred immunostimulatory active component for use in this invention. An IgG protein contains (1) a Fab region (including the VH, VL and CH₁ domains); (2) a hinge region, and (3) an Fc region (including the CH₂ and CH₃ domains). The Fab region is the region of an antibody protein which includes the antigen-binding portions. The “hinge” region is a flexible area on the immunoglobulin polypeptide that contains many residues of the amino acid proline and is where the Fc fragment joins one of the two Fab fragments. The Fc region is the constant region on an immunoglobulin polypeptide; is located on the immunoglobulin heavy chains; and is not involved in binding antigens. The Fc region can bind to an Fc receptor on phagocytes. The amino-proximal end of the CH₂ domain, especially amino acids 234 to 237, is important for binding of the Fc region to the Fc receptor. Fc receptors, such as Fc RI, are integral membrane proteins located on phagocytic white blood cells, such as macrophages. The hinge region is important for regulating Fc-Fc receptor interactions, providing flexibility to the polypeptide and functioning as a spacer.

The immunoglobulin used for immunostimulatory component can be obtained from any vertebrate, such as human or mouse. Preferably, the polynucleotide encodes an immunoglobulin having a substantial number of sequences that are of the same origin as the host. For example, if a human is treated with a complex of the invention, preferably the immunoglobulin is of human origin.

Therefore, it is to be appreciated that preferably, the IgG is generated in the same species as the subject affected by said pathologic agent.

IgG Fc associated to solid support (or any other optional connecting component) in the reverse orientation, retains the biological function of IgG Fc of binding Fc receptor to mediate macrophage activation. Therefore it is to be noted that the IgG Fc, should project away from the connecting component of the complex of the invention, in a “reversed orientation”, thus mimicking the configuration of IgG during opsonization. This IgG which serves as the active component binding the Fc receptor to activate phagocytes, such as macrophages, and may also activate the complement cascade (“complement fixation”).

Phagocytes respond to signals from the Fc receptors by assembling cytoskeletal proteins, signaling cytoskeletal-protein assembly by activation of protein tyrosine kinases, and by “phagocytosing” the cell coated with immunoglobulin. IgG-Fc RI interaction activates various biological functions such as phagocytosis, endocytosis, ADCC, release of inflammatory mediators and superoxide anion production. Macrophages possess organic anion transporter proteins that promote the afflux of anionic substances from the macrophage. Thus, Fc RI mediates ADCC by macrophages and triggers both phagocytosis and superoxide production. For that reason, the complexes of the invention where the Fc domain of IgG is associated as an active component, interact with phagocyte Fc receptor and cause phagocytes to bind to the complex bound to the target pathologic agent (through its targeting component), inducing ADCC. The IgG₁ and IgG3 isotypes, that interact with the high affinity receptor Fc RI on macrophages, are preferred for the complexes and methods of the invention.

Macrophages, dendritic cells and B cells are all antigen presenting cells (APC), which can also present antigens to T cells. In this way, APCs are involved in other components of the immune response, including the humoral immune response (antibody production) and cellular immune response. Thus, APCs which engulf the complex of the invention which stimulates binding and entrance of the pathogen via Fc receptor, present the antigenic determinants of the target agent to T cells, stimulates cellular and humoral immune response specific for the target agent. This particular possibility is especially advantageous in viral infections where no effective therapy is available. Therefore, the complex of the invention may be used as a specific therapy in viral infections.

According to anther preferred embodiment, the optional connecting component comprised within the targeting complex of the invention, may be any one of a solid support such as polystyrene beads, Sepharose beads, agarose beads, cellulose beads, a chemical linker, a peptide, oligopeptide or polypeptide linker, a saccharide, oligosaccharide or polysaccharide linker, poly-amine and lipid linker and any protein or particularly scaffold protein.

As indicated above, both active and targeting components may be associated by a chemical linker to the optional connecting component. Alternatively, the chemical linker itself may function as the connecting component. Cross-linking proteins is well known in the art, see e.g., Chemistry of Protein Conjugation and Cross-linking, Shan S. Wong, CRC Press, 1991. Proteins may be cross-linked by their functional groups. Usually, the SH or NH2 groups of proteins are used for that purpose. Chemical groups that react with SH groups include e.g., dithio groups, including pyridyldithio groups, haloacetamido groups, including iodoacetamido groups, maleimido groups, including alkylmaleimido groups, and the like groups known to the skilled person. Amino groups may be coupled using optionally sulfonated N-hydroxysuccinimide ester groups, imidoester groups, including methyl pimelimidate and methyl suberimidate groups, or carbodiimide groups. Also free carboxyl groups of a protein may be used for cross-linking, e.g. using an amino group such as an alkylamino group, and providing a dehydrating agent in the reaction.

The cross-linker used for proper association of both complex components may be homobifunctional or heterobifunctional. Examples for homobifunctional cross-linkers include disuccinimidyl suberate (DSS), disuccinimidyl glutarate (DSG) and dimethyl suberimidate (DMS). Examples for heterobifunctional cross-linkers include m-maleimideobenzoyl-N-hydroxysuccinimide ester (MBS) and N-gamma-maleimidobutyryloxy-succinimide ester (GMBS). In yet another embodiment of the invention, a cross-linker is capable of reacting unspecifically with proteins, for instance by photoactivation. Examples for photoreactive groups are e.g., the azidobenzoyl, azido-nitrobenzoyl, azido-hydroxybenzoyl or azido-coumarin groups. Examples of photoreactive cross-linkers include p-nitrophenyl-2-diazo-3,3,3-trifluoropropionate (PNP-DTP) and azidobenzoyl hydrazide.

A carbohydrate-reactive cross-linker may be also used. Carbohydrate reactive groups include e.g., the aldehyde group, the glyoxal group, or the sulfone group. Cross-linkers reactive with carbohydrates include e.g., the above azidobenzoyl hydrazide, 4-[m-maleimidomethyl]-cyclohexane-1-carboxyl-hydrazide (M₂C₂H), or 4-(4-N-maleimidophenyl)-butyric acid hydrazide (MPBH). If any of the complex components (active, targeting and optionally connecting component) does not comprise cysteine residues, a photoreactive cross-linker or an amino-reactive cross-linker may be used, such as the activated N-hydroxy succinimide derivative of the above M₂C₂H or MPBH, e.g., 4-(4-(succinimido-N-oxo)-phenyl)-butyric acid hydrazide. Alternatively, when it is desired to use the above carbohydrate and sulfhydryl-reactive cross-linkers, a second cross-linker may be used, which may be linked to the sulfhydryl-reactive moiety of the first cross-linker. The protein may then be coupled via the second functionality of the second cross-linker, which advantageously is a group reactive with amino groups, such as an activated N-hydroxy succinimide ester group.

The above noted cross-linkers are commercially available, e.g., from PIERCE, as listed at p. O-90 to O-104 of the 1994 Life Sciences Product Catalog and Handbook of PIERCE, Rockford, Ill. 61105 USA, or from other suppliers in the field of organic chemistry, such as e.g., Sigma, St. Louis, USA.

U.S. Pat. No. 5,399,501 describes the conjugation of immunologically active proteins, e.g. antibodies, to a solid phase via a rather elaborate set of three distinct molecules: first, a cross-linker which binds to amino, carboxyl or thiol groups on the surface of the solid phase and provides a group capable of reacting with thiols (e.g. maleimide); second, a cross-linker that binds to NE₂ groups of the protein to be conjugated and also provides a group capable of reacting with thiols (e.g. maleimide), and third, a dithiol reagent capable of joining the solid-phase bound thiol-reactive group with the protein-bound thiol-reactive group. This set of cross-linkers may also be used in the present invention, for the purpose of cross-linking of the targeting component (e.g., IgY) and the active component (IgG) together, or associating both components to a connecting component (such as a solid support, beads, etc. ).

In another embodiment of the invention, the active component or the targeting component may be cross-linked directly to the connecting component that may be for example a solid support comprising carbohydrates. This may be accomplished e.g., by using a bifunctional cross-linker capable of reacting with carbohydrates on the one hand, and with proteins on the other. For instance, the cross-linker azidobenzoyl hydrazide (ABH) may be coupled to the active or the targeting component, using the photoactivatable azide group of the cross-linker to nonspecifically bind to said component. In a second step, this “activated” component may then be reacted with the solid support, whereby the carbohydrate-reactive hydrazide group of the cross-linker binds to glucose units in the carbohydrate. This non-covalent bond may then be allowed to form, depending upon the reaction conditions, such as concentration of salt, presence of caustic solutions, reaction time, and the like, to form the desired complex.

Association of both components may be carried out for example using a peptide linker as the connecting component. The peptide linker is a peptide of suitable amino acid sequence which is expected not to interfere with the secondary and tertiary structure of both components. The linker peptide may be connected to the active or the targeting component by a cross-linker, as described above for linking proteins. The necessary functional groups for cross-linking may be provided in the linker peptide by the choice of amino acids. For instance, lysine or arginine is chosen when it is desired to use amino groups for cross-linking. cysteine residues are chosen when it is desired to use sulfhydryl groups for cross-linking. Glutamic acid or aspartic acid may be chosen when it is desired to use carboxylic acid groups for cross-linking. Groups that are not desired to be reacted may be protected by a suitable protection group as known in the art for amino, carboxyl, or sulfhydryl groups.

The linker may preferably comprise between 10 and 150 amino acids in length. Further preferably, the linker comprises small, uncharged amino acids, such as glycine, alanine, valine, serine, or threonine. The linker preferably contains glycine and serine residues. The ratio between the glycine and serine residues is preferably about 3:1 to 4:1. An example for a 17 amino acid Glycine/Serine linker is GGGGSGGGGSGGGGSGG.

Alternatively, both components may be linked via a peptide, by using recombinant DNA technology.

In one preferred embodiment, association of both active and target component through a solid support, may be preformed by the avidin-biotin system, as described in a large number of publications. The avidin molecule is a protein originally isolated from chicken egg protein. This protein may be expressed as a fusion protein with one of the complex components, or preferably may be cross-linked to the connecting component (solid support, such as beads). The other component may then be cross-linked to biotin, which is a small vitamin molecule. Methods for cross-linking biotin to proteins are well known to the person of skill in the art, and have been widely published in many articles and text books. In yet another option, the connecting component may be an avidin molecule or any fragment thereof. In such case, the targeting component should be biotinylated and the immunoactive component would be an anti avidin IgG antibody. It is to be appreciated that other high affinity systems may be used for associating the complex of the invention. Such systems include as non-limiting example the GST-glutathione system and CBD-cellulose.

In another preferred embodiment, the complex of the invention comprises only target component and immunoactive component. Both components are directly attached without any connecting component. According to this particular embodiment, the target component may be an antibody targeted against the target agent, for example an IgY antibody, and the active component would be an IgG anti IgY antibody. Binding of both components retains the appropriate conformation (having the Fc of the IgG molecule free).

It is to be appreciated that any targeting complex of the invention bay be comprised of different targeting components targeted to different fragments of a target pathogen or to different pathogens.

In a particular embodiment, the invention relates to a multi-functional targeting complex that induces specific phagocytosis of at least one target mastitis pathogenic bacteria in cattle infected by said bacteria. This specific complex comprises: (a) at least one target recognition component comprising a molecule which specifically binds to the mastitis pathogenic bacteria or to any functional fragment of said bacteria; (b) at least one immunoactive component comprising phagocytosis inducing agent; and (c) optionally, a connecting component that associates said at least one targeting component and said at least one active component. Both active and targeting components are associated, optionally by the connecting component, in an appropriate conformation which preserves the independent biological functions of both components.

This targeting complex according to a specific embodiment comprises as a target recognition component, an IgY antibody specific for mastitis pathogenic bacteria. More specifically, the targeting complex of the invention may comprise at least one IgY antibody specific for at least one of the mastitis pathogenic bacteria such as, but not limited to, Actinomyces, E. coli, Pseudomonas, Clostridium, Staphylococcus aureus, Streptococcus agalactiae, Streptococcus dysgalactiae, Corynebacterium pyogenes, Enterococcus faecalis, Corynebacterium bovis, Nocardia asteroides (or farcinica) and Streptococcus uberis, mycoplasina, viruses and endotoxins.

Optionally, said complex may comprise different IgY antibodies, each targeted to a different mastitis pathogenic bacteria.

According to a specifically preferred embodiment, the targeting complex of the invention comprises as a phagocytosis-inducing agent an IgG molecule preferably generated in a bovine animal.

In another specific embodiment, the invention relates to a multi-functional targeting complex for inducing specific phagocytosis of at least one target pathogenic bacteria causing ulcerative disease in fish infected by said bacteria, said complex comprises: (a) at least one target recognition component comprising a molecule which specifically binds to Aeromonas spp. pathogenic bacteria which causes ulcerative disease in fish, or to any functional fragment of said bacteria; (b) an active component comprising phagocytosis inducing agent; and (c) optionally, a connecting component that associates at least one of said targeting component and at least one of said active component, wherein the independent biological functions of said targeting component and said active component are retained.

The particular targeting complexes of the invention may optionally further comprise as a connecting component, any one of, but not limited to, a solid support, a chemical linker, polypeptide linker, protein, polysaccharide linker and lipid linker.

Such solid support may be for example a polystyrene micro-sphere bead, and more particularly, an avidin-coated polystyrene bead. In this particular case, the target recognition component may be a biotinylated IgY antibody specific for mastitis pathogenic bacteria. Further, this particular targeting complex may preferably comprise as the phagocytosis-inducing agent, an anti-avidin IgG molecule, which is most preferably, generated in a bovine animal.

In a further aspect, the invention relates to a composition for the treatment of a pathologic disorder in a subject. The composition of the invention comprises as active ingredient a multi-functional targeting complex in accordance with the invention.

The composition of the invention may optionally further comprise pharmaceutically and/or veterinarily acceptable diluent, excipient and/or additive.

According to one embodiment, the composition of the invention is intended for the treatment of a pathological disorder that may be any one of immune-related disorders, viral, fungal or bacterial infections and malignant disorders, in a subject in need of such treatment.

The pathological disorder to be treated may be a viral infection caused by different viruses such as influenza virus, human immunodeficiency virus, Epstein-Barr virus, cytomegalovirus, vaccinia virus, hepatitis virus, mouth and foot disease, Newcastle disease virus and herpes virus, or a malignant disease such as melanoma, carcinoma, lymphoma and sarcoma.

The subject to be treated may be according to a preferred embodiment, any one of human, domestic and non-domestic mammal, domestic bird, aquaculture, preferably, fish and exotic aquarium fish. It should be appreciated that the treated subject may be also any reptile or zoo animal. More preferably, the composition of the invention is intended for domestic mammals. By “mammalian subject” is meant any mammal for which the proposed therapy is desired, including human, equine, canine, feline subjects, and most preferably, cattle, particularly bovine animals such as cows, goats and sheep. In yet another specific embodiment, the complex and composition of the invention is intended for the treatment of domestic fish and exotic fish such as for example, carp and Koi fish.

In a particular embodiment, the pathologic disorder to be treated with the composition of the invention may be a bacterial infection. More preferably, the bacterial infection may be bovine mastitis.

Therefore, the invention further provides a composition for the treatment of mastitis in bovine animals. Such specific composition according to one embodiment comprises as active ingredient a multi-functional targeting complex that induces specific phagocytosis to at least one target mastitis pathogenic bacteria in bovine infected by said bacteria. More specifically, the complex comprised within this specific composition for mastitis, comprises: (a) at least one target recognition component comprising a molecule which specifically binds to said mastitis pathogenic bacteria or any functional fragment of said bacteria; (b) an active component comprising phagocytosis-inducing agent; and (c) optionally, a connecting component that associates at least one of said targeting component and at least one of said active component. It is to be noted that by association of both components, optionally by the connecting component, the independent biological functions of the targeting component and the active component are preserved.

According to a specifically preferred embodiment the composition of the invention comprises as an active ingredient, a complex comprising an avidin-coated polystyrene bead as a connecting component, a biotinylated IgY antibody specific for mastitis pathogenic bacteria, as the targeting component, and an anti-avidin IgG molecule, which is most preferably, generated in a bovine animal.

The invention further provides for a composition for the treatment of ulcerative disease in fish. Such specific composition comprises as active ingredient a multi-functional targeting complex, that induces specific phagocytosis to at least one target Aeromonas spp. pathogenic bacteria in fish infected by said bacteria, said complex comprises: (a) at least one target recognition component comprising a molecule which specifically binds to said Aeromonas spp. pathogenic bacteria or any functional fragment of said bacteria, preferably, such component may be IgY antibody which specifically recognizes antigenic determinants on said bacteria; (b) an active component comprising phagocytosis inducing agent, preferably, an immunoglobuline molecule generated in fish; and (c) optionally, a connecting component that associates at least one of said targeting component and at least one of said active component, wherein the independent biological functions of said targeting component and said active component are retained.

The present invention further relates to a composition for inducing a specific phagocytosis targeted to at least one target pathologic agent related to a pathological disorder in a subject suffering from said disorder. According to one embodiment, this composition comprises as an active ingredient, the multi-functional targeting complex of the invention, and optionally further comprises pharmaceutically and/or veterinarily acceptable diluent, excipient and/or additive.

According to a specific embodiment the invention relates to a composition for inducing a specific phagocytosis of at least one target mastitis bacterial pathogen in a female bovine animal suffering from mastitis. This composition comprises as an active ingredient, a complex comprising an avidin-coated polystyrene bead as a connecting component, a biotinylated IgY antibody specific for mastitis pathogenic bacteria, as the targeting component, and an anti-avidin IgG molecule, which is most preferably, generated in bovine and optionally further comprises pharmaceutically and/or veterinarily acceptable diluent, excipient and/or an active or supporting additive.

In yet another particular embodiment, the invention further provides for a composition for inducing a specific phagocytosis of at least one target Aeromonas spp. bacterial pathogen in a fish suffering from ulcerative disease. Such specific composition comprises as an active ingredient a multi-functional targeting complex according to the invention, and optionally further comprises pharmaceutically and/or veterinarily acceptable diluent, excipient and/or additive.

In a third aspect, the present invention relates to a method for the treatment of a pathological disorder in a subject. The method of the invention comprises administering to the subject a therapeutically effective amount of a targeting complex according to the invention or of a pharmaceutical composition comprising the same.

As used herein, “effective amount” means an amount necessary to achieve a selected result. For example, an effective amount of the composition of the invention useful for inducing targeted immuno-stimulatory effect, and preferably, phagocytosis towards the target pathologic agent.

The complex of the invention may be administered directly to the subject to be treated, or it may be desirable to administer to the subject compositions comprising said targeting complex and it may be desirable to add acceptable carriers, adjuvants or diluents to the composition prior to its administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the subject. Formulations include those suitable for oral, rectal, nasal, or parenteral (including subcutaneous, intramuscular, intravenous, intramammary and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

The compositions of the invention may further contain a pharmaceutically acceptable carrier, additive, diluent or excipient. Suitable carriers include, e.g., saline phosphate buffered saline, and saline with 5% HSA or PPF. Other suitable carriers are well known to those of skill in the art and are not a limitation on the present invention. Similarly, one of skill in the art may readily select other desired components for inclusion in a pharmaceutical composition of the invention, and such components are not a limitation of the present invention.

The pharmaceutical compositions of the invention generally further comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The composition of the invention may be mixed with nutritive feed material or water supplies for the animal. It is contemplated however that the effective composition can either be mixed with the nutritive feed material or water or fed to the animal separately. The effective composition must be provided in an amount effective to stimulate targeted immuno-stimulatory reaction and preferably, phagocytosis towards the pathologic agent.

More preferably, the complex or compositions of the invention may be administered by a route selected from oral, intravenous, parenteral, transdermal, subcutaneous, intravaginal, intranasal, mucosal, sublingual, topical and rectal administration and any combinations thereof. For the treatment of mastitis, topical intramammary administration is preferred. For the treatment of ulcerative disease, particularly, furculosis in fish, topical or intraperitoneal administration are preferred. It should be noted that preferred topical treatment of fish may be performed by dipping the fish in water containing the specific complex of the invention.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

In one embodiment, the method of the invention utilizes any one of the compositions according to the invention. More preferably, these compositions comprising as an effective ingredient any one of the targeting complexes of the invention.

In yet another embodiment, the method of the invention is intended for the treatment of a pathological disorder which may be any one of immune-related disorders, viral, fungal or bacterial infections and malignant disorders.

In a specifically preferred embodiment, the pathological disorder may be a bacterial infection.

In a particularly preferred embodiment, the invention relates to a method for the treatment of mastitis in bovine. This specific method comprises administering to said bovine in need a therapeutically effective amount of a targeting complex capable of inducing specific phagocytosis targeted to at least one target mastitis pathogenic bacteria in said infected bovine, or of a pharmaceutical composition comprising the same, said complex comprises: (a) at least one target recognition component comprising a molecule which specifically binds to said pathologic agent or to any functional fragment of said agent; (b) an active component comprising an immuno-stimulatory agent; and (c) optionally, a connecting component that associates at least one of said targeting component and at least one of said active component. The association of said targeting component and said active component, optionally by said connecting component, should retain the independent biological functions of both the active and the targeting components.

The invention further provides a method for stimulating specific phagocyte activity. A targeting complex containing the IgG is made to contact a phagocyte, such as a macrophage or other APC. The contact stimulates an increased phagocytic activity by the phagocyte. The phagocyte may engulf the complex bound to the target agent and enables rapid clearance of said pathologic agent from the host.

In yet another particularly preferred embodiment, the method of the invention utilizes a particular composition of the invention. According to this embodiment, this composition comprises as an active ingredient, a complex comprising an avidin-coated polystyrene bead as a connecting component, a biotinylated IgY antibody specific for mastitis pathogenic bacteria, as the targeting component, and an anti avidin IgG molecule, which is most preferably, generated in bovine. This particular composition may optionally further comprise pharmaceutically and/or veterinarily acceptable diluent, excipient and/or additive.

Still further, the invention relates to a method for the treatment of ulcerative disease in fish comprising administering to said fish in need a therapeutically effective amount of a targeting complex capable of inducing specific phagocytosis targeted to at least one target Aeromonas spp. pathogenic bacteria in said infected fish according to the invention, or of a pharmaceutical composition comprising the same.

The present invention further relates to the use of a multi-functional targeting complex of the invention, in the preparation of a pharmaceutical composition for the treatment of a pathological disorder in a subject in need.

This pathological disorder may be according to a specific embodiment any one of immune-related disorders, viral, fungal or bacterial infections and malignant disorders. Preferably, the pathologic disorder is a bacterial infection.

In another specifically preferred embodiment, the invention relates to the use of a specific multi-functional targeting complex, in the preparation of a pharmaceutical composition for the treatment of mastitis in bovine. This particular complex comprising an avidin-coated polystyrene bead as a connecting component, a biotinylated IgY antibody specific for mastitis pathogenic bacteria, as the targeting component, and an anti avidin IgG molecule, which is most preferably, generated in bovine.

In yet another embodiment, the invention relates to the use of another particular multi-functional targeting complex, in the preparation of a pharmaceutical composition for the treatment of mastitis in bovine. Such complex comprises a targeting component and an immunoactive component, both are directly attached without any connecting component. According to such embodiment, the targeting component may be an IgY antibody directed against mastitis pathogenic bacteria, and the immunoactive component is an IgG anti IgY antibody. Binding of both components retains the appropriate conformation (having the Fc of the IgG molecule free).

Unless defined otherwise, all technical and scientific terms used herein have the same meaning as commonly understood by one of ordinary skill in the art to which this invention belongs. All patents and publications cited in this specification are incorporated by reference.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Bacteria

Specific mastitis pathogens (Staphylococcus aureus, Streptococcus agalactiae, Streptococcus dysgalactiae, Streptococcus uberis, and E. coli) were either purchased from ATCC (Hy Lab, Rehovot, Ill.) or taken from stocks of the Israeli Veterinary Authorities (Beit Dagan, Ill.), according to the following accession numbers; Staphylococcus aureus, 2449/1; E. coli, Ein Hashofet 169. ATCC: Streptococcus agalactiae ATCC 27956, Streptococcus dysgalactiae ATCC 27957, Streptococcus uberis ATCC 27958.

Antigen Preparation

Bacteria as well as purified proteins were used as antigens for the production of specific IgY. Bacteria were cultured in a specific medium as indicated below, and were prepared for vaccination as follows;

Freeze dried bacteria (Staphylococcus aureus, Streptococcus agalactiae, dysgalactiae and uberis) were thawed [1 ml Nutrient broth (NB) (Difco, Detroit, Mich.)], inoculated on blood agar and incubated at 37° C. for 24 h to confirm vitality and purity. 3-4 colonies were selected and inoculated in 250 ml 37° C. Columbia broth (CB) (Difco, Detroit, Mich.) (12-24 h at 37° C.) supplemented with 0.1% D-glucose, yeast extract, and 0.5% NaCl in a 1 liter Ehrlenmeyer and incubated at 37° C. for 10 h. Streptococcus were inoculated in 250 ml NB at 37° C. The bacteria were separated by centrifugation (1000×g, 15 min) and the pellet was washed (×3) and resuspended in non-pyrogenic PBS (pH 7.6; 0.01M) to approximate 1.2×10⁹ cells, as indicated by McFarland equivalence turbidity standard 4 (1.2×10⁹ cell/ml) (remal, 12076 Santa Fe, Lenexa, Kans.). To confirm purity, blood agar plates were inoculated and incubated. The pellet was subjected to mechanical agitation with glass beads by cell homogenizer (Braun Melsungen A G, Germany). After removal of the glass beads and intact bacteria, the homogenate was filtered through 0.2 μm-pore-size membranes, protein concentrations were determined with the Protein Assay kit (Bio-Rad, UK) and stored at −20° C. To confirm sterility, blood agar plates were inoculated and incubated under appropriate conditions.

In an additional method, bacteria were inoculated and allowed to grow as described above. After incubation, bacteria were harvested by centrifugation at 10,000×g for 20 minutes and then suspended in 50 mM PBS (pH 7.0). Bacteria were then divided into three groups for the following processes: (1) inactivation with formaldehyde (0.05%), (2) inactivation by heat (100° C. for 10 minutes), and (3) sonication. A mixture of the treated fractions was used for vaccination.

Preparation of E. coli Antigen

E. coli were cultured in 250 ml NB (Difco, Detroit, Mich.)], in a 1 liter Ehrlenmeyer and incubated at 37° C. for 24 h. The bacteria pellet was sonicated with glass beads as described above.

Preparation of the Targeting Component

Production of IgY

Three to six, 35-50 wk-old commercial White Leghorn hens per group were immunized by an intramuscular injection at two sites of breast muscle of 0.05-05-0.5 mg of antigen with Freund's incomplete adjuvant, in a total volume of 0.8-1.0 ml per chicken. The adjuvant comprised half of the injected volume. A booster injection was given intramuscularly 2-3 wk thereafter with the same dose emulsified with Freund's incomplete adjuvant. Blood samples were collected from wing vein on every week after the initial injection. Eggs were collected daily and stored below 20° C. until analyzed.

Isolation of IgY from Egg Yolk

IgY were isolated from by the following steps:

1. Separation of the yolk from the white;

2. Water dilution of yolks (10 ml yolk to 100 ml water), pH=6.8 and stirring with an electric mixer in temperature less than 20 C for 4-6 hrs;

3. Delipidation—by filtration of the soluble water fraction under vacuum through a filter paper (1574 Runfilter, Schleicher & Schuell GmbH, Germany).

4. Precipitation of the soluble filtered proteins by ammonium sulfate (60%; w/v)

5. Centrifugation (10000 g, 20 min) and re-suspending the precipitate in ammonium sulfate (60% w/v) medium at 4° C.

6. Isolation of the IgY by gel filtration, using T-gel (Pierce, USA), anion-exchange (DEAE-Sephacel) and gel filtration (Ultrogel AcA 22) techniques.

7. The crude (5) or the purified (6) IgY were diluted in a serial 1:10 dilution order with PBS before use.

IgY Specificity

Since most commercial birds are routinely vaccinated against NDV (New Castle Disease), anti-NDV antibodies were used to test the efficiency of IgY isolation. This was done by hemagglutination inhibition test.

ELISA for Intact Bacteria

Quantitative ELISA for measurement of specific anti-bacteria IgY concentrations and Standard IgY: Wells in a plate were coated overnight with 100 μl of polylysine (10 μg/ml), then 200 μl of specific bacteria in broth (0.3 OD) were added. Twenty four hrs later, 100 μl of gluataraldehyde (0.07%) were added for 15 min and washed with PBS. Wells were blocked with 100 μl of 2% milk powder and 100 mM glycine. Crude samples of IgY were serialy diluted in PBS (Akita and Nakai, 1992) and 100 μl of serial 1:10 dilution in duplicates were applied to the wells. Plates were incubated at 37° C. for 2 h and washed with PBS-T (Phosphate Buffer Saline—TWEEN20 ™ and then incubated with 100 μl of rabbit anti-chicken IgY conjugated with horseradish peroxidase (1:10000 in PBS). After incubation at 37° C. for 1 h, plates were washed three times with PBS-T (Phosphate Buffer Saline—TWEEN 20), followed by addition of 100 μl of freshly prepared substrate solution, 2-2-azino-bis (3-ethylbenzthiazoline-6-sulfonic acid) in 0.05 M phosphate citrated buffer (pH 5.0). The optical density at 450 nm was then recorded.

IgY and Bacteria Growth Pattern-Agglutination Test

In order to examine the extent of out-membrane antigen recognition by IgY and its inhibitory effect on bacterial growth, various bacterial pathogens [10⁷ CFU/ml (colony forming units) E-media broth] were cultured in a tripticase soy broth (TSB) in the presence of specific- and non-specific control IgY. IgY concentration ranged between 250-500 μg/ml were incubated at 37° C. for 1 to 3 h. The bacteria growth curve was calculated using optical density measurement at 590 nm.

Antigen-Antibody Binding and Pathogen Growth Pattern

The effectiveness of IgY to bind, neutralize or reduce the proliferation of various mastitis pathogenic bacteria was examined in an additional method. Mastitis pathogen (10⁷ CFU/ml E-media broth) was cultured in TSB with specific- and non-specific control IgY (250-500 μg/ml) and allowed to insulate at 0-4° C. for 30 min. Then cultures were diluted to serial dilutions with a sterile TSB and 10 μl (in triplicates) of the diluted medium, were inoculated on the appropriate plate and were incubated at 37° C. for 24 hrs. The colonies of each treatment were counted and their size was measured.

Micro/Nano Carrier Preparation

Commercial carboxylated microbeads are activated by using 1-Ethyl-3-(3-dimethylaminopropyl)carbodiimide hydrochloride (EDC) in the presence of Avidin. The EDC activate the carboxyls group and allowed efficient coupling of amine group in avidin with carboxyl group. This results in formation of amide bonds coupling avidin molecules to each micro-bead.

Attachment of the Targeting component to the Activated Beads

At the first stage, IgY antibody (specific anti E. coli IgY for example) is biotinylated using Sulfo-NHS-biotin reagent using common biotinylation method. The resulting biotinylated anti E. coli IgY is then mixed with Avidin-microbeads at various ratio for 1 hr at room temp. This step is followed by addition of Bovine anti Avidin antibody which allowed to reacted with the microbead-Avidin-Biotinylated-IgY. In this context, antibody was raised in calf immunized with Avidin.

Development of an In-vitro Assay for Phagocytosis

The present procedure was adapted from the TECHNICAL DATA SHEET 430 (2001) of PolySciences Inc (PA, USA).

Polystyrene microparticles (1, 2 and 3 μm) have been used for phagocytosis studies. To elicit this response, the particles were first coated with avidin, then a specific bovine IgG anti-avidin and the specific IgY produced against mastitis pathogens were bound, as described above. Microbeads (1 um, 1×10⁶ per group), were coated with various ratios of IgG:IgY (0.6 to 0.7 μg/ml, 60 min 37 C). After opsonization, one million microbead coated components were incubated with or without 10⁷ E. coli bacteria and were exposed to cow blood phagocytes (granulocytes, monocytes, and macrophages). Cells were continuously mixed for 60 min at 37° C. during which phagocytosis occurs. The reaction was then stopped by the addition of ice cold PBS, centrifuged 1000×g for 5 min and samples of 10 μl diluted in a 1:10 order were applied on a tryptic soy agar plate for determination of CFU. Then the cells were washed to remove any free particles in the medium and re-suspended in cold deionized H₂O and analyzed for the number of bacteria. This procedure enables the determination of phagocytic activity by measuring the number of bacteria left in medium after incubation with known numbers of bacteria with the described complex.

Phagocytosis Assay with Multi-Functional Targeting Complex (Microbead Coated Components)

(1) 200 μl of anticoagulated whole blood were added to a polypropylene tube.

(2) 10 μl of microbead coated components (10⁸ particles/ml) were added to the tube and incubated with gentle shaking for 30 to 60 min at 37° C.

(3) At the end of the incubation phagocytosis is terminated by adding 2 ml of ice cold PBS and mixing.

(4) The cell pellet is re-suspended in 3 ml of sterile water and gently mixed for 20 to 30 seconds. 1 ml of 3.5% NaCl is added to make the suspension isotonic and the cells are pelleted by centrifuging at 500×g for 5 min.

(5) The cells were resuspended in 500 μl of cold PBS, and samples were kept at 4° C., and analyzed as soon as possible.

SCC (Somatic Cell Counts)

The SCC was determined with a Coulter cell counter (CC), Z1 model, Coulter Electronics Limited, Luton, UK).

N-acetyl- -D-glucosaminidase (NAGase)

The concentration of NAGase in milk was fluorometrically determined according to the ADLMILK NAGase test, (ADC Applied Diagnostics Corporation, Helsinki, Finland) with a computerized microplate setting. A value of 100 units corresponds to a release of about 5 μmol of product per liter per minute at 25° C.

ELISA Procedure for Measuring IgY in Milk

Quantitative ELISA for measurement of specific anti-bacteria IgY concentrations in milk: Wells of ELISA plate were coated overnight with 100 μl of Rabbit-anti-IgY-Fc, then were blocked for 1 hr in 37° C. with 200 ul of 2% milk buffer. Volume of 100 μl the tested solution or commercial IgY (used for standard curves) was added to the ELISA plates and incubated for 1 hr in 37° C., followed by five washes with PBS plus Tween 20 (0.1%). Plates were then incubated with 100 μl of rabbit anti-chicken IgY conjugated with horseradish peroxidase (1:1000 in PBS). After incubation at 37°C. for 1 h, plates were washed five times with PBS-T (Phosphate Buffer Saline—TWEEN 20), followed by addition of 100 μl of freshly prepared substrate solution, 2-2-azino-bis(3-ethylbenzthiazoline-6-sulfonic acid) in 0.05 M phosphate citrated buffer (pH 5.0).

### Example 1

Preparation of Targeting Component Targeted Against Bovine Mastitis Bacterial Pathogens

Immune Response against Bovine Mastitis E. Coli

IgY antibodies specific for bovine mastitis E. Coli were prepared as detailed in experimental procedures. The effectiveness of specific anti-pathogen IgY in prevention or treatment of bovine mastitis, was next examined.

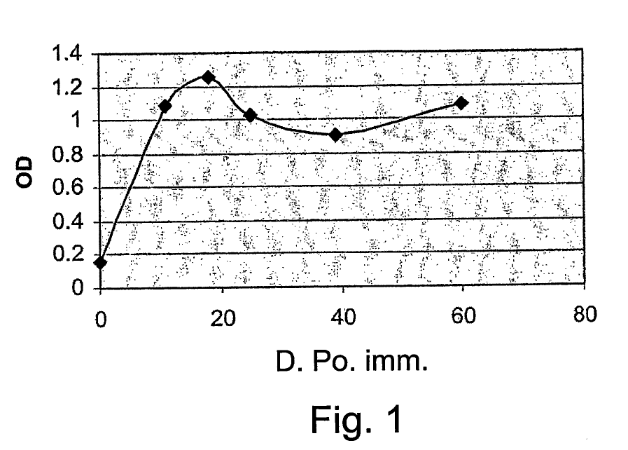

The activity of antibody (OD of the crude sodium sulfate egg yolk precipitate diluted by 1:1000) against mastitis intact E. Coli was determined by ELISA using the egg yolk preparation from laying hens as shown in FIG. 1. The pre-immune antibody activity was below 0.2 OD, increased rapidly 3 weeks later and reached a plateau thereafter.

Immune Response Against Bovine Mastitis Staphylococcus aureus

The activity of antibody (diluted by 1:100) against mastitis intact Staphylococcus aureus, that was prepared according to experimental procedures, was determined by ELISA as described above.

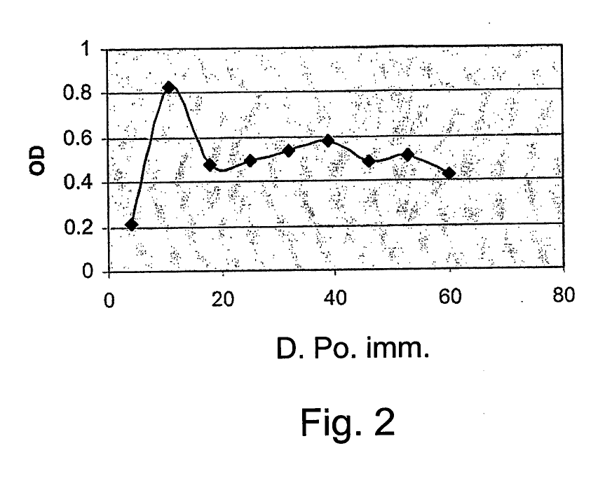

As shown in FIG. 2, antibody activity increased markedly one-week post immunization, then decreased about half way down and reached a plateau thereafter. However the titer was lower than that found against E. coli.

Inmune Response Against Bovine Mastitis Streptococcus uberis

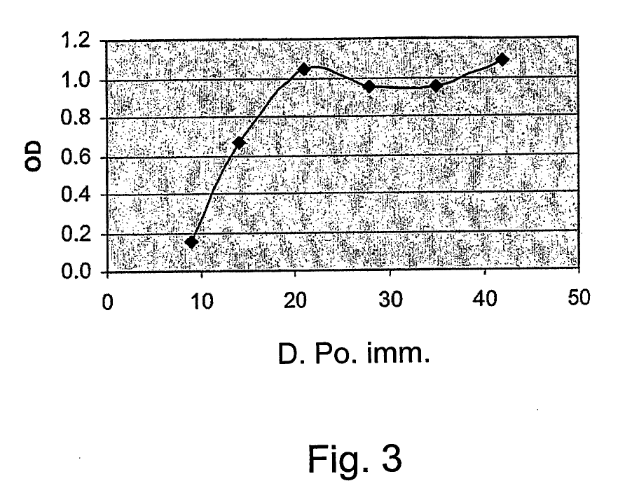

The activity of antibody (diluted by 1:1000) against mastitis intact Streptococcus uberis was determined by ELISA as described above. Antibody activity increased markedly two-week post immunization and reached a plateau thereafter, as shown by FIG. 3.

### Example 2

Agglutination of Mastitis Pathogen Growth using IgY Specific Antibodies

Agglutination Assay of Mastitis E. Coli

In order to evaluate the extent of out-membrane antigen recognition by IgY and its inhibitory effect on bacterial growth, various bacterial pathogens (10⁷ CFU/ml E-media broth) were cultured in a tripticase soy broth (TSB) in the presence of specific- and non-specific control IgY, and were incubated for 1 to 3 h at 37° C. The IgY concentration ranged between 250-500 μg/ml. The growth curve of the bacteria was calculated by optical density measurements of viable bacteria in culture at 590 nm.

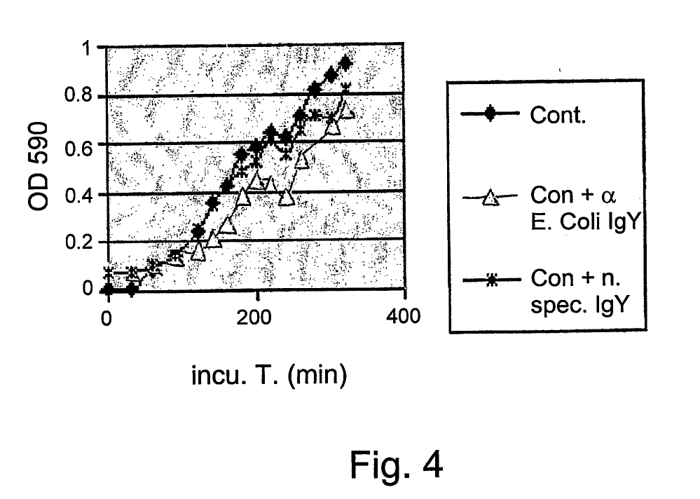

As shown by FIG. 4, E. Coli (107 CFU/ml) cultures in TSB were incubated in the presence of 436 μg/ml of anti-E. coli IgY (open triangles), 436 μg/ml of non specific IgY (asterisk) and control (black diamonds) IgY at 37° C. and the optical density of the culture was measured at OD 590 nm through 6 h post incubation. Results indicated that incubation of cultures in the presence of the specific IgY derived against E. Coli, led to about 20% growth inhibition. Non-specific IgY had minor effect on growth pattern.

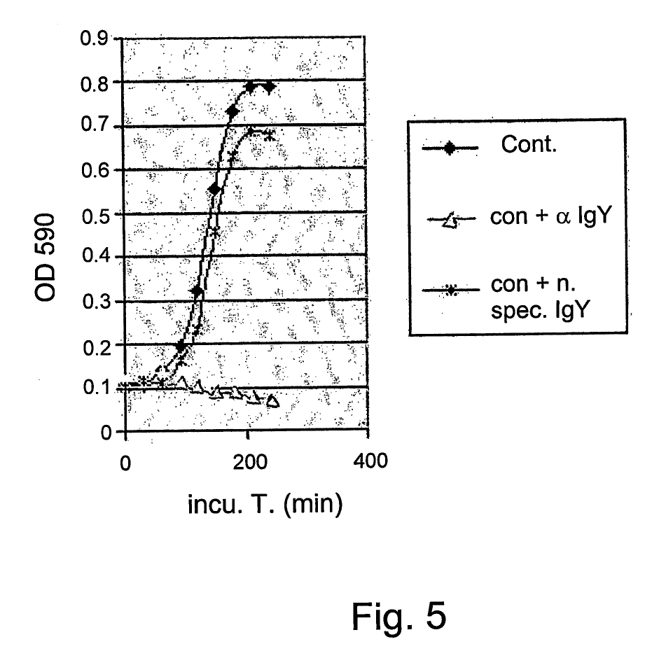

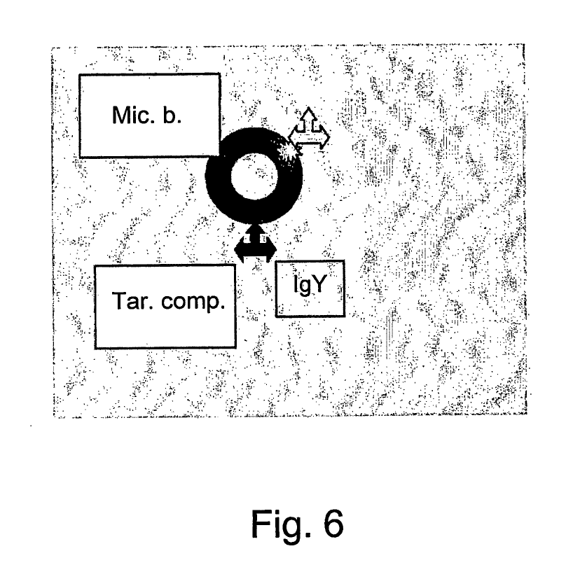

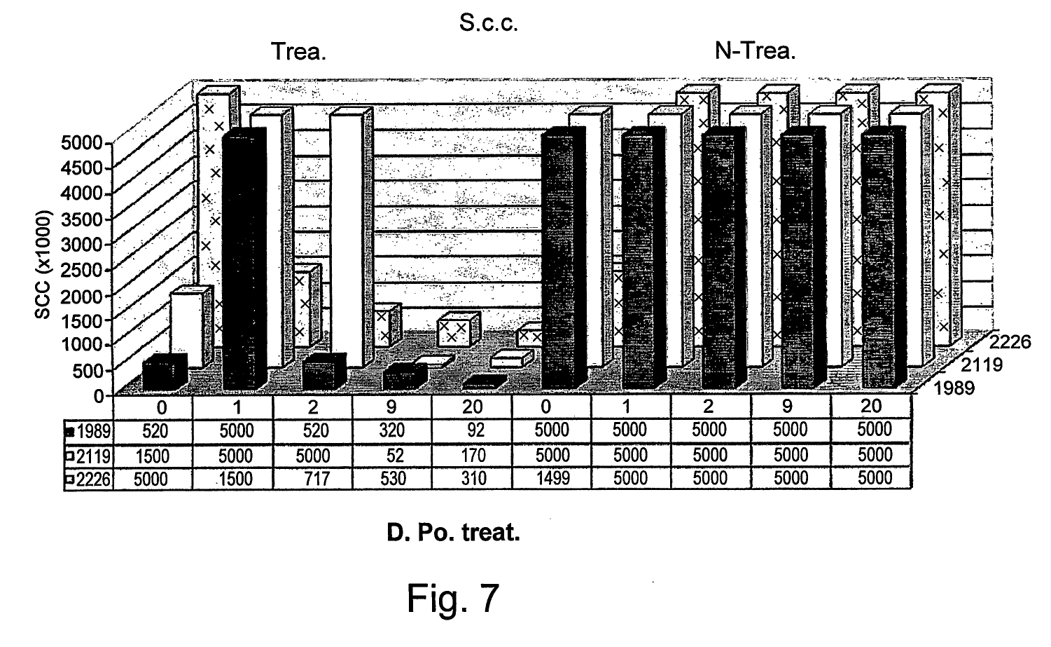

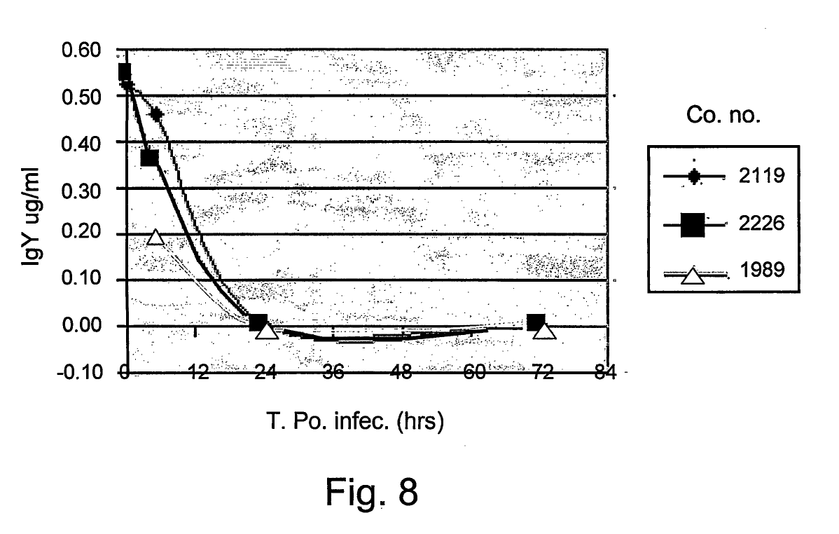

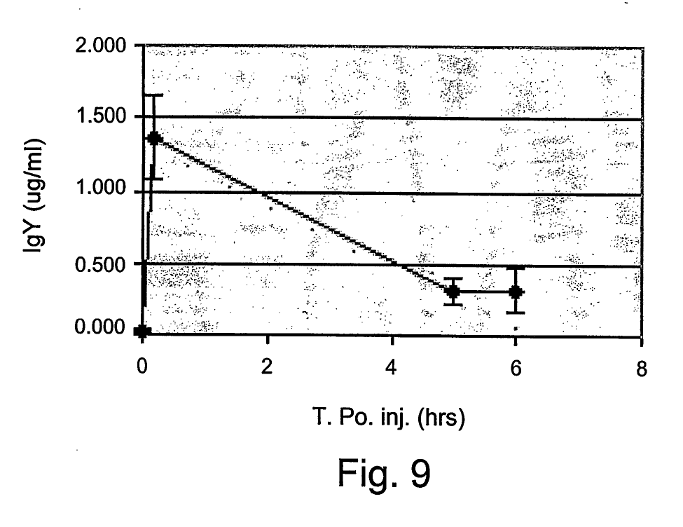

In order to optimize conditions for bacterial growth inhibition, by the targeting component of the invention, cultures were incubated in the presence of increasing concentrations of specific anti E. coli IgY. As shown by FIG. 5, E. coli culture (1×10⁷ CFU/ml) in TSB was incubated with 650 μg/ml of 0.5 U (units) filtrated anti-E. coli-IgY (open triangles), with 650 μg/ml of non specific IgY (asterisks) and control (black diamonds) IgY at 37° C. and the optical density of the culture was measured at OD 590 nm through 6 h post incubation.

Results indicated that the specific IgY completely inhibited the growth of E. coli. The non-specific control IgY had only a minor effect on bacteria agglutination and growth. These results indicate that the developed IgY anti E. coli serves as a useful tool for prevention of mastitis caused by E. coli.

Antigen-Antibody Binding and Pathogen Growth Pattern

The effectiveness of IgY to bind, neutralize or reduce the proliferation of E. coli was further examined. Starter culture of mastitis E. coli (10⁷ CFU/ml E-media broth) was cultured at 4-8° C. for 30 min with: (a) control TSB medium+PBS; (b) control TSB medium+pre-immune non-specific IgY; (c) control TSB medium+specific IgY harvested after 8 weeks and (d) control TSB medium+specific IgY harvested 10 weeks after first boost.

The results summarized in Table 1 demonstrate that growth of the negative control E. coli yielded about 5×10⁷ CFU/ml E-media broth, and control with non-specific IgY about 6×10⁷ CFU. Treatments (c) and (d), which were pre-cultured with the specific anti-E. coli IgY, resulted in about 90% growth inhibition of the control. These results indicate that the developed IgY well recognizes mastitis E. coli, and enables its binding and inhibition of its growth pattern.

TABLE 1
Growth and development of E. coli (CFU/10 ul media
broth) pre-cultured (0-4° C.) with or without
specific anti E. coli IgY and inoculated onto an
agar plate.

| | CFU/10 ul media broth, |
|---|---|
| Treatments | as % of control |
| (a) control TSB medium + PBS | 100 |
| (b) control TSB medium + pre- | 100 |
| immune non-specific IgY | |
| (c) control TSB medium + | 12 |
| specific IgY harvested 8 weeks | |
| after first boost | |
| (d) control TSB medium + | 9 |
| specific IgY harvested 10 weeks | |
| after first boost | |

Effectivity of IgY from different immunization batches, on binding and neutralization of E. coli, as well as the and antibody levels were examined in a further experiment. Starter mastitis E. coli (10⁷ CFU/ml E-media broth) was cultured at 4-8° C. for 30 min with: (a) control TSB medium+PBS; (b) treatment (a)+pre-immune non-specific IgY; (c) treatment (a)+specific IgY harvested 2 weeks post-immunization; (d) treatment (a)+specific IgY harvested 4 weeks post-immunization; (e) treatment (a)+specific IgY harvested 5 weeks post-immunization; (f) treatment (e) with double amount of specific IgY harvested 5 weeks post-immunization; and (g) treatment (a)+specific IgY harvested 7 weeks post-immune. As shown by Table 2, IgY was found to be capable of binding and inhibiting E. coli growth similarly at fferent batches of egg collections, and increasing IgY levels further inhibited bacterial growth (e vs. if).

TABLE 2
Growth and development of E. coli (CFU/10 ul media broth)
pre-cultured (4-8° C.) with or without specific anti
E. coli IgY and inoculated in an agar plate.

| | CFU/10 ul |
|---|---|
| | media broth, |
| Treatments | as % of control |
| (a) control TSB medium + PBS; | 100 |
| (b) (a) + pre-immune non- | 100 |
| specific IgY; | |
| (c) (a) + specific IgY harvested 2 | 15 |
| weeks post-immunization; | |
| (d) (a) + IgY harvested 4 weeks | 14 |
| post-immunization; | |
| (e) (a) + IgY harvested 5 weeks | 19 |
| post-immunization; | |
| (f) (e) with double the amount of | 16 |
| specific IgY; | |
| (g) (a) + IgY harvested 7 weeks | 18 |
| post-immunization; | |

### Example 3

Attachment of the Targeting Component to a Micro/Nano Carrier Bead

The different targeting components prepared as described in Example 1, specifically for different mastitis bacterial pathogens, were next attached to microbeads. Commercial carboxylated microbeads are activated by using 1-ethyl-3-(3-dimethylaminopropyl)carbodiimide hydrochloride (EDC) in the presence of Avidin. The EDC couples the carboxyls to primary amine residues in Avidin resulting in formation of amide bonds and in coupling of Avidin molecules to the activated microbeads.

IgY antibody (specific anti mastitis pathogenic bacteria IgY, anti E. coli) is biotinylated using Sulfo-NHS-biotin reagent using common biotinylation method.

Biotinylated anti E. coli IgY is mixed with Avidin-microbeads at various ratios for 1 hr at room temp.

In order to attach the active component to the complex of the invention, bovine anti Avidin antibody, raised in calf immunized with Avidin, was reacted with the microbead-Avidin-Biotinylated-IgY. The binding is due to the affinity of the bovine antibody to Avidin. Binding of the anti Avidin antibody to the Avidin covered beads, is performed through the Fab fragment of the antibody, and therefore this part of the molecule is projecting towards the bead, while the Fc fragment projects away from the bead surface, as required for efficient opsonization.

### Example 4

Phagocytosis Assay using the Multi-Functional Targeting Complex of the Invention (Microbead Coated Components)

The ability of the targeting complex of the invention to induce direct phagocytosis against a pathogenic target was next examined. In a first study, approx. 6.5×10⁶ E coli mastitis bacteria were incubated with fresh (2-3 hrs) cow blood phagocytes and with microbeads coated with various ratios of IgY (anti different mastitis bacteria, as a targeting component) and IgG (as the immunoactive component) components for 60 min at 37 C.

Most bacteria were engulfed during the 60 min of incubation period, however the number of bacteria that escaped phagocytosis was related to ratio of IgY:IgG. The highest phagocytosis rate was observed with the ratio of 0.9:0.1 for IgY to IgG respectively.

Results of Table 3 confirm the facilitated engulfing of bacteria by macrophage.

TABLE 3
Induction of phagocytosis by the complex of the invention

| | IgY:IgG | | | | | |
|---|---|---|---|---|---|---|
| | 1/0 | 0.9/0.1 | 0.7/0.3 | 0.5/0.5 | 0/1 | 0/0 |
| Number of bacteria | 145 | 0 | 145 | 290 | 290 | 435 |
| escaped | | | | | | |
| phagocytosis | | | | | | |
| in percent | 33% | 0% | 33% | 67% | 67% | 100% |
| Phagocytosis rate | 67% | 100% | 67% | 33% | 33% | 0% |

In a further study approx. 3×10⁶ E. coli mastitis bacteria were incubated with fresh blood phagocytes and with microbeads coated with various ratios of IgY:IgG components for 60 min at 37° C.

Approximately 70% of bacteria were engulfed during the 60 min of incubation period, however the number of bacteria that escaped phagocytosis was again related to ratio of IgY:IgG. The highest phagocytosis rate was observed with the increased ratio of IgY in the microbead.

The effect of the phagocytes on the viability of the bacteria in the presence of the complex of the invention was also examined.

For testing the viable bacteria, cells were centrifuged (1000×g), washed and then plated on appropriate plates in increasing dilutions. Viable colonies were counted. As shown by Table 4, only few bacteria survived phagocytosis (between 1300 to 16000) in the presence of the targeting complex of the invention, whereas 1.5×10⁶ bacterial cells were counted in culture that was not exposed to the targeting complex of the invention.

TABLE 4

| | 1 | 2 | 3 | 4 | 5 |
|---|---|---|---|---|---|
| IgY:IgG ratio | 1/0 | 0.9/0.1 | 0.7/0.3 | no | no |
| | | | | microbead | phagocyte |
| | | | | control | control |
| Number of | 9425 | 21025 | 34800 | 275500 | 1450000 |
| bacteria leftafter | | | | | |
| phagocytosis | | | | | |
| Number of | 0.7% | 1.5% | 2.4% | 19% | 100% |
| bacteria escaped | | | | | |
| phagocytosis | | | | | |
| Phagocytosis rate | 99.3% | 98.5% | 97.6% | 81% | 0% |
| (vs. no. 5) | | | | | |
| Phagocytosis due | 97% | 92% | 87% | 0% | |
| to microbead (vs. | | | | | |
| no. 4) | | | | | |

### Example 5

In vivo Treatment of Mastitis caused by Infection of Streptococcal Dysgalactiae using the Targeting Complex of the Invention

As indicated in the background, Streptococcal dysgalactiae, gram-positive bacteria, are one of the mastitis causing bacteria. These bacteria can exist in the udder between 20 to 30 days and may cause clinical or subclinical mastitis, leading to economical losses due to decreased milk production, lost milk quality premiums due to increased SCC (somatic cell count), and changes in milk composition. Many mastitis pathogens (Strep. agalactiae and Staph. aureus) have been recently evacuated from Israeli herds, however an increase in the occurrence of other non-agalactiae species was noted.

Therefore, the inventors have next examined the effectiveness of the targeting complex of the invention in in-vivo treatment of Strep. dysgalactiae infections. These in vivo studies were performed in the Veterinary Institute in Beit-Dagan Israel. Two quarters of four Holstein medium-yielding-milking cows (25 L/d) were intramammarily injected with Strep. dysgalactia (1750 CFU of STRPVL1860 in a volume of 5 ml per quarter). Cows were clinically evaluated, three days prior to and after the inoculation, for mastitis using the following parameters: flakes or clots in the milk; hot, swollen quarter or udder; fever; rapid pulse; and loss of appetite. In addition to clinical evaluation, the Afifarm v 2.03 system was used for measuring milk production, activity and milk conductivity, and milk samples taken were analyzed for bacteria, quantitative SCC and NAGase activity. As shown by Table 5, all measured parameters indicated that the infected tested cows suffered from mastitis.

Forty-eight hours after the infection with bacteria, positive bacterial cultures (Strep. dysgalactia) were found in all injected quarters (Table 5). An increased level of SCC and NAGase activity were also noted (except for cow 265 [B/R] that showed only mild increase).

TABLE 5
Bacteriology, Somatic Cell Counts (SCC) and NAGase
activity in milk of cows, 48 hours post intramammary
injection with Strep. dysgalactia (1750 CFU of STRPVL1860
in a volume of 5 ml per quarter).

| NAGase | | | Sampling | | |
|---|---|---|---|---|---|
| activity | SCC | Bacteriology | time | | |
| (U) | (×1000) | S. dysgalactia | (hours) | Quarter | Cow |
| 2 ± 2 | 125 ± 35 | − | −1 | B/L | 265 |
| 366 | 5000< | + | 24 | | |
| 128 | 5000< | + | 48 | | |
| 3 | 170 ± 41 | − | −1 | B/R | |
| 26 | 256 | − | 24 | | |
| 14 | 590 | + | 48 | | |
| 28 ± 11 | 147 ± 100 | − | −1 | F/L | 1989 |
| 113 | 436 | + | 24 | | |
| 85 | 237 | + | 48 | | |
| 25 ± 7 | 210 ± 72 | − | −1 | F/R | |
| 550 | 5000< | + | 24 | | |
| 351 | 5000< | + | 48 | | |
| 5 ± 3 | 120 ± 32 | − | −1 | B/L | 2119 |
| 25 | 313 | + | 24 | | |
| 22 | 134 | + | 48 | | |
| 6 ± 7 | 129 ± 33 | − | −1 | B/R | |
| 577 | 5000< | + | 24 | | |
| 253 | 5000< | + | 48 | | |
| 18 ± 7 | 210 ± 55 | − | −1 | B/L | 2226 |
| 32 | 715 | + | 24 | | |
| 325 | 5000< | + | 48 | | |
| 13 ± 9 | 142 ± 47 | − | −1 | B/R | |
| 54 | 162 | + | 24 | | |
| 297 | 1499 | + | 48 | | |

F—Fore;
B—back;
L—left;
R—right;
+/− (positive/negative) Streptococcus dysgalactia

Seventy-four hours after Strep. dysgalactiae inoculation, the left quarter of each of the four tested cows was treated with the targeting complex of the invention having a target recognition component comprising an IgY antibody specific for Strep. dysgalactiae [half of the quarters were treated by injection of 20 (cows 1989, 2226) or 50 (cows 265, 2119) million units/quarter each in 5 ml of pyrogen-free saline]. Two intra-mammary injections were performed in the treated quarter of each of the four tested cows (one injection after morning milking and a second injection 24 hrs later). The right quarters were untreated and served as control. Milk samples were monitored during the experimental period (three weeks).

In three out of four non-treated quarter of the tested cows, S. dysgalactia bacteria were found in milk samples throughout 20 days of experiment, indicating that infection proceeded into its chronic phase. SCC level and NAGase activity were very high in the three quarters, however in cow 265 the level of these parameters returned to their initial level with no bacteria in milk (Table 6).

As shown by Table 6 and FIG. 7, regardless the dose given, injection of the anti S. dysgalactia specific complex of the invention to half of the treated quarters, caused a significant decrease in the number of bacteria in three out of four cows (Table 6), while in controls the quarters were infected throughout 20 days of study.

The results of this study clearly indicate that the anti S. dysgalactia specific complex of the invention efficiently reduced the number of bacteria in milk of infected cows. Furthermore, SCC and NAGase activity were decreased significantly after 24 to 48 hours in at least 3 out of 4 cows (Table 6 and FIG. 7). It can be seen that one week after treatment was given, the treated quarters were completely recovered from mastitis, whereas control quarters were highly infected (Table 6).

TABLE 6
Bacteriology, SCC and NAGase activity in milk of four infected
cows, after two-consecutive intramammary injection of 20 and 50
million units of the S. dysgalactia specific complex of the
invention per quarter).

| | | | Sampling | | | |
|---|---|---|---|---|---|---|
| NAGase | SCC | Bacteriology | time | Y-Complex | | |
| activity | (×10³) | S. dysgalactia | (days) | Units × 10³ | Quarter | Cow |
| 289 | 5000< | + | 0 | 50,000 | B/L | 265 |
| 310 | 5000< | + | 1 | 50,000 | | |
| 376 | 5000< | + | 2 | 0 | | |
| 98 | 5000< | + | 9 | 0 | | |
| 109 | 5000< | + | 20 | 0 | | |
| 14 | 590 | + | 0 | 0 | B/R | |
| 16 | 500 | + | 1 | 0 | | |
| 17 | 302 | + | 2 | 0 | | |
| 9 | 195 | − | 9 | 0 | | |
| 7 | 219 | − | 20 | 0 | | |
| 102 | 520 | + | 0 | 20,000 | F/L | 1989 |
| 136 | 5000< | + | 1 | 20,000 | | |
| 84 | 520 | + | 2 | 0 | | |
| 49 | 320 | − | 9 | 0 | | |
| 25 | 92 | − | 20 | 0 | | |
| 303 | 5000< | + | 0 | 0 | F/R | |
| 248 | 5000< | + | 1 | 0 | | |
| 489 | 5000< | + | 2 | 0 | | |
| 467 | 5000< | + | 9 | 0 | | |
| 267 | 5000< | + | 20 | 0 | | |
| 75 | 1500 | + | 0 | 50,000 | B/L | 2119 |
| 98 | 5000< | − | 1 | 50,000 | | |
| 274 | 5000< | − | 2 | 0 | | |
| 8 | 52 | − | 9 | 0 | | |
| 11 | 170 | − | 20 | 0 | | |
| 279 | 5000< | + | 0 | 0 | B/R | |
| 153 | 5000< | + | 1 | 0 | | |
| 654 | 5000< | + | 2 | 0 | | |
| 776 | 5000< | + | 9 | 0 | | |
| 278 | 5000< | + | 20 | 0 | | |
| 325 | 5000< | + | 0 | 20,000 | B/L | 2226 |
| 69 | 1500 | + | 1 | 20,000 | | |
| 50 | 717 | − | 2 | 0 | | |
| 25 | 530 | − | 9 | 0 | | |
| 21 | 310 | − | 20 | 0 | | |
| 297 | 1499 | + | 0 | 0 | B/R | |
| 81 | 5000< | + | 1 | 0 | | |
| 111 | 5000< | + | 2 | 0 | | |
| 157 | 5000< | + | 9 | 0 | | |
| 475 | 5000< | + | 20 | 0 | | |

F—Fore;
B—back;
L—left;
R—right;
+/− (positive/negative) Streptococcus dysgalactia

Toxicity Test of the Anti S. dysgalactiae Specific IgY Complex of the Invention

Toxicity of anti S. dysgalactiae specific IgY complex was next studied, using commercial healthy Holstein dairy cows that were injected with different amounts of the complex of the invention. Four lactating cows were selected after visual checking for inflamed quarters, and monitoring their bacteriology profile in milk (free of bacteria, NAGase<30; SCC<150,000).

Three to ten million units of the anti S. dysgalactiae specific IgY complex in 5 ml pyrogen-free sterile saline solution were intra-mammary injected into the tested quarter using a sterile plastic syringe.

Clinical examination was then performed in the treated cows (flakes or clots in the milk; slight swelling of infected quarter; hot, swollen quarter or udder; fever; rapid pulse; and loss of appetite) as well as chemical measurements in milk sampled during 3 days post injection.

No clinical symptoms or any behavioral changes were observed in cows with the treated quarters

As shown by Table 7, milk analysis indicated that injection of the anti S. dysgalactiae specific IgY complex of the invention at a level of 3×10⁶ units per quarter did not dramatically affect the level of the SCC. The SCC increased slightly after 24 hours but returned to the normal level 24 hours later. Similar results were obtained when NAGase activity at level of 3 or 10 million units of anti S. dysgalactiae specific IgY complex. These findings indicate that the complex of the invention has only minor effect on the mammary tissue and milk in response to injection.

TABLE 7
NAGase activity and somatic cell counts (SCC, average ± SD)
during 72 hours after intra-mammary injection of Y-Complex in
four cows at levels of 3 or 10 millions units per quarter

| | SCC (×1000) | | | | NAGase activity (Units) | | | |
|---|---|---|---|---|---|---|---|---|
| Complex | [Time post injection (hrs)] | | | | [Time post injection (hrs)] | | | |
| (Units) | 6 | 24 | 48 | 72 | 6 | 24 | 48 | 72 |
| 3 × 10⁶ | 70 ± 96 | 363 ± 511 | 66 ± 56 | 70 ± 96 | 1 ± 1 | 9 ± 10 | 18 ± 32 | 0 ± 0 |
| 10 × 10⁶ | ND | ND | NM | NM | 87 ± 58 | 73 ± 60 | 23 ± 16 | NM |

NM—Not Measured;
ND—Non detectable

Residue of the Anti S. dysgalactiae Specific IgY Complex in Milk

To further examine the safety of using the specific S. dysgalactiae complex of the invention, milk samples obtained from treated cows, were monitored for the existence of traces of this complex. Milk samples taken at 0, 10 min, 5, 24, 48 and 72 hours after injection of the complex, were subjected to specific ELISA as described in experimental procedures, for determining the levels of the IgY. As shown by FIG. 8, the level of IgY in milk (free and bound to microsphere beads) decreased sharply after injection. Ten minutes after injection, only between about 0.5 to 0.6 μg IgY per ml milk was detected. These levels decreased rapidly, and no IgY was detected 24 hours after injection. It should be noted that the minimal detected level was 6.25 ng/ml.

Similarly, analysis of samples taken up to 8 hr post injection clearly indicated that the concentration of IgY decreased by 80% 5 hours after injection and after one milking (FIG. 9).

### Example 6

In vivo Treatment of Mastitis Caused by Infection of E. coli using the Targeting Complex of the Invention

The inventors next examined the effectiveness of the targeting complex of the invention in in-vivo treatment of another mastitis causing bacteria, E. coli. These in vivo studies were also performed in the Veterinary Institute in Beit-Dagan Israel.

Four non-pregnant Israeli-Holstein cows in their 1^{st}-4^{th} mid-lactation period were included in two consecutive trials, two cows each. The cows were chosen for the experiment according to their udder condition as confirmed by three consecutive bacteriological and somatic cell count (SCC) examinations of quarter milk samples. The cows were free of udder infection and had SCC lower than 120×10³ cells/ml. The cows were milked three times daily and had milk yields of 25-35 kg/day. Food was offered in mangers located in free-stall barns.

The E. coli strain (P4) was grown in Nutrient Broth (Difco, Detroit, Mich.) for 24 h at 37° C. and harvested by centrifuging at 3000×g for 15 min at 4° C. The pellet was resuspended in non-pyrogenic PBS (pH 7.6; 0.01M) and washed, and the bacterial concentration determined by serial dilution on blood-agar plates. The original pellet was stored on ice for 16 h. Each cow was challenged by injection of 5 ml of the suspension, containing 500 colony-forming units (CFU), into two quarters (frontal or rear) through the streak canal into the gland cistern.

Duplicate quarter milk samples were obtained according to the International Dairy Federation (IDF) (1985) procedures and submitted to the laboratory within 1 h. Bacteriological analysis was performed according to accepted standards. A 0.01-ml aliquots of each milk sample were spread over blood-agar plates (Bacto-Agar; Difco Laboratory) containing 5% sheep red blood cells. All plates were incubated at 37° C. and examined for growth at 18 and 42 h. Gram negative colonies were identified with the API 20 E or API NE kit (bioMerieux S. A., 69280 Marcy-l'Etoile, France).

Prior to this assay the tested cows were tested for bacteriological status, NAGase activity and SCC, on three consecutive days. Cows were brought to the stalls 1 to 2 h after milking and their teats were washed with warm water, dried, disinfected with individual non-woven towelettes moistened with chlorhexidine, cetrimide and ethanol (Medi-Wipes, A L Baad, Massuot Itzhak, Israel). The first three milk squirts were discarded and samples (50 ml/quarter) were then taken into a sterile tube for bacteriology, SCC, and NAGase activity. Each cow was intrammamrlly inoculated with 500 cfu/quarter in tow quarters, frontal or rear immediately to morning milking. In the 48-h post-challenge period, cows were closely monitored for symptoms of illness (rectal temperature, heart and respiratory rate, alimentary tract activity (rumen contraction) and udder temperature, pain, edema and udder size. At 6, 30 and 54 (only in trial 1) hour post-challenge quarters were sampled, cows were milked and the left challenged quarter of each cow was treated. Cows were not milked at the following milking (night) and returns to milking scheme after (morning). Quarters were continued to be sampled every 24-48 hour up to 15 days post challenge. Cows were intrammamarlly treated with 40×10⁶ units of Complex (anti E. coil), given with suspension in a 5 ml non-pyrogenic PBS.

As shown by FIG. 10A, E. coli bacteria were found in milk samples throughout 15 days of experiment, indicating that infection proceeded into its chronic phase. However, in milk samples obtained from the treated quarters, clear decrease in bacterial infection was shown. Significant protection of the E. coli specific complex was also observed when SCC level and NAGase activity were examined (FIGS. 10B and 10C, respectively).

These results clearly indicate that the anti E. coli specific complex of the invention efficiently reduced the number of bacteria and SCC in milk of infected cows and efficiently recovered infected quarters and protects cows from mastitis.

### Example 7

In vivo Treatment of Carps Suffering of Ulceration caused by Infection of Aeromonas salmonicida. using the Targeting Complex of the Invention

The effective use of different targeting complexes of the invention in the treatment of mammalian diseases, e.g., mastitis in cattle, led the present inventors to examine the possibility of treating non-mammalian animals, particularly, fish, using the complex of the invention. Thus, the inventors have developed a specific complex targeted against Aeromonas salmonicida which causes furunculosis in Koi pet fish.

Aeromonas salmonicida is a Gram-negative bacterial pathogen that is the causal agent of furunculosis in salmonid fishes, a debilitating and lethal disease encountered in aquaculture. Current vaccines offer limited effectiveness and epizootics are common in farmed fish. Development of broadly effective control measures requires a more complete understanding of the interactions between pathogen and host factors that contribute to the disease state.

The following experiments were performed using carps infected with Aermonas salmonicida as a model system. Four tanks (0.5 m³ each) supplied with fresh water throughout the experiment (flowthrough system), were used for this experiment. Each tank contains thirty healthy carps (30 gr each) and five ulcerated fish (Koi) that were kept together for twenty-eight days (cohabitation). The ulcerated Koi were then removed, and ten to fourteen days after cohabitation the infected carps were treated using the Aeromonas salmonicida specific complex of the invention which comprises as a targeting component a specific IgY antibody directed against Aeromonas salmonicida. Each of the thirty fish in two of the four tanks that were used as the test treated groups is injected twice intraperitoneally (10 to 14 and at day 17 to 19) with 0.5 ml of solution containing the complex of the invention diluted 1:5 [3 mg IgY/kg body weight (BW) or 450 million of complex units per kg/BW] in saline. Fish of the two control tanks are treated with saline and are monitored similarly to the test two tanks, for the development of clinical signs associated with ulcerative disease of carp.

At the end of the study described above, fish of the control groups were divided into two groups. Complex (450 million U/kg BW) of the invention comprises as a targeting component a specific IgY antibody directed against Aeromonas, was added to the water of the treated group while the other group was untreated.

1. A multi-functional targeting complex for inducing a specific immuno-stimulatory reaction against at least one target pathogenic agent, said complex comprises:
a. at least one target recognition component comprising a molecule which specifically binds to said target agent or any functional fragment of said agent;
b. an immunoactive component comprising an immuno-stimulatory agent; and
c. optionally, a connecting component that associates said at least one targeting component and said at least one immunoactive component, wherein the independent biological functions of said targeting component and said active component are retained.

2. The targeting complex according to claim 1, wherein said immuno-stimulatory reaction is selected from the group consisting of phagocytosis, antibody-dependent cell-mediated cytotoxicity (ADCC), complement activation, and stimulation of specific T cells and B cells.

3. The targeting complex according to claim 2, wherein said immuno-stimulatory reaction is phagocytosis by leukocyte.

4. The targeting complex according to claim 1, wherein said target pathologic agent is selected from the group consisting of bacteria, viruses, fungi, malignant cells, toxins, venoms, haptens, environmental pollutants and undesired proteins.

5. The targeting complex according to claim 4, wherein said target pathologic agent is bacteria selected from the group consisting of mastitis pathogenic bacteria, enteropathogenic bacteria, pathogenic furonculosis bacteria and ulcerative disease causing bacteria.

6. The targeting complex according to claim 5, wherein said bacteria is at least one mastitis pathogenic bacteria selected from the group consisting of Actinomyces, E. coli, Pseudomonas, Clostridium, Staphylococcus aureus, Streptococcus agalactiae, Streptococcus dysgalactiae, Corynebacterium pyogenes, Enterococcus faecalis, Corynebacterium bovis, Nocardia asteroides and Streptococcus uberis.

7. The targeting complex according to claim 5, wherein said ulcerative disease causing bacteria is Aeromonas spp. which causes ulcerative disease in fish.

8. The targeting complex according to claim 1, wherein said target recognition component comprises any one of antibody or any functional fragments thereof, ligand and receptor specific for said pathologic agent or any functional fragment of said agent.

9. The targeting complex according to claim 8, wherein said target recognition component comprises an antibody or any functional fragments thereof specific for said pathologic agent or any functional fragment of said agent, said antibody being selected from the group consisting of IgG, IgY and IgM.

10. The targeting complex according to claim 9, wherein said recognition component comprises an IgY antibody or any functional fragment thereof specific for said pathologic agent or any functional fragment of said agent.

11. The targeting complex according to claim 1, wherein the immunoactive component comprises an immuno-stimulatory agent which induces phagocytosis.

12. The targeting complex according to claim 11, wherein said phagocytosis inducing agent is selected from the group consisting of opsonins which are any one of IgG and C3b, protein molecules having carbohydrate residues that interact with the mannose-fucose receptor of phagocytes, ligand protein molecules recognized by receptors on scavenger macrophages, ligands for integrins expressed by phagocytes, glycoproteins and fucosyl transferase.

13. The targeting complex according to claim 12, wherein said phagocytosis inducing agent is IgG.

14. The targeting complex according to claim 1, wherein the connecting component is any one of a solid support, a chemical linker, polypeptide linker, polysaccharide linker and lipid linker.

15. A multi-functional targeting complex for inducing specific phagocytosis of at least one target mastitis pathogenic bacteria in bovine infected by said bacteria, said complex comprises:
a. at least one target recognition component comprising a molecule which specifically binds to said mastitis pathogenic bacteria or any functional fragment of said bacteria;
b. an active component comprising phagocytosis inducing agent; and
c. optionally, a connecting component that associates at least one of said targeting component and at least one of said active component, wherein the independent biological functions of said targeting component and said active component are retained.

16. The targeting complex according to claim 15, wherein said target recognition component is an IgY antibody specific for at least one mastitis pathogenic bacteria.

17. The targeting complex according to claim 16, wherein said mastitis pathogenic bacteria is at least one bacteria selected from the group consisting of Actinomyces, E. coli, Pseudomonas, Clostridium, Staphylococcus aureus, Streptococcus agalactiae, Streptococcus dysgalactiae, Corynebacterium pyogenes, Enterococcus faecalis, Corynebacterium bovis, Nocardia asteroides and Streptococcus uberis.

18. The targeting complex according to claim 17, wherein said phagocytosis inducing agent is an IgG molecule generated in bovines.

19. The targeting complex according to claim 18, wherein the connecting component is any one of a solid support, a chemical linker, polypeptide linker, polysaccharide linker and lipid linker.

20. A multi-functional targeting complex for inducing specific phagocytosis of at least one target pathogenic bacteria causing ulcerative disease in fish infected by said bacteria, said complex comprises:
a. at least one target recognition component comprising a molecule which specifically binds to Aeromonas salmonicida. pathogenic bacteria which causes ulcerative disease in fish, or to any functional fragment of said bacteria;
b. an active component comprising phagocytosis inducing agent; and
c. optionally, a connecting component that associates at least one of said targeting component and at least one of said active component, wherein the independent biological functions of said targeting component and said active component are retained.

21. A composition for the treatment of a pathologic disorder in a subject, comprising as an active ingredient a multi-functional targeting complex that induces in said subject a specific immuno-stimulatory reaction to (against) at least one target pathologic agent, said complex comprising:
a. at least one target recognition component comprising a molecule which specifically binds to said pathologic agent or any functional fragment of said agent;
b. an active component comprising an immuno-stimulatory agent; and
c. optionally, a connecting component that associates said at least one targeting component and said at least one active component, wherein the independent biological functions of said targeting component and said active component are retained.

22. The composition according to claim 21, wherein said composition optionally further comprises pharmaceutically and/or veterinarily acceptable diluent, excipient and/or additive.

23. The composition according to claim 22, wherein said pathological disorder is any one of immune-related disorders, viral, fungal or bacterial infections and malignant disorders.

24. The composition according to claim 23, wherein said complex is according to any one of claims 2 to 19.

25. The composition according to any one of claims 19 to 22, wherein said subject affected by the pathogenic agent is any one of human, domestic mammal, domestic bird, domestic fish, pet and exotic fish.

26. The composition according to claim 25, wherein said domestic mammal is bovine.

27. The composition according to claim 26, wherein said pathologic disorder is a bacterial infection.

28. The composition according to claim 27, wherein said bacterial infection is mastitis.

29. A composition for the treatment of mastitis in bovine, comprising as active ingredient a multi-functional targeting complex, that induces specific phagocytosis to at least one target mastitis pathogenic bacteria in bovine infected by said bacteria, said complex comprises:
a. at least one target recognition component comprising a molecule which specifically binds to said mastitis pathogenic bacteria or any functional fragment of said bacteria;
b. an active component comprising phagocytosis inducing agent; and
c. optionally, a connecting component that associates at least one of said targeting component and at least one of said active component, wherein the independent biological functions of said targeting component and said active component are retained.

30. The composition according to claim 29, wherein said complex is according to any one of claims 17 to 19.

31. The composition according to claim 25, wherein said subject is any one of domestic fish, pet and exotic fish.

32. The composition according to claim 31, wherein said bacterial infection is Aeroinonas spp. infection which causes ulcerative disease in said fish.

33. A composition for the treatment of ulcerative disease in fish, comprising as active ingredient a multi-functional targeting complex, that induces specific phagocytosis to at least one target Aeromonas spp. pathogenic bacteria in fish infected by said bacteria, said complex comprises:
a. at least one target recognition component comprising a molecule which specifically binds to said Aeromonas spp. pathogenic bacteria or any functional fragment of said bacteria;
b. an active component comprising phagocytosis inducing agent; and
c. optionally, a connecting component that associates at least one of said targeting component and at least one of said active component, wherein the independent biological functions of said targeting component and said active component are retained.

34. A composition for inducing a specific phagocytosis targeted to at least one target pathogenic agent related to a pathological disorder in a subject suffering from said disorder, which composition comprises as an active ingredient a multi-functional targeting complex according to any one of claims 1 to 20, and optionally further comprises pharmaceutically and/or veterinarily acceptable diluent, excipient and/or additive.

35. A composition for inducing a specific phagocytosis of at least one target mastitis bacterial pathogen in a bovine suffering from mastitis, which composition comprises as an active ingredient a multi-functional targeting complex according to any one of claims 17 to 19, and optionally further comprises pharmaceutically and/or veterinarily acceptable diluent, excipient and/or additive.

36. A composition for inducing a specific phagocytosis of at least one target Aeromonas spp. bacterial pathogen in a fish suffering from ulcerative disease, which composition comprises as an active ingredient a multi-functional targeting complex according to claim 20, and optionally further comprises pharmaceutically and/or veterinarily acceptable diluent, excipient and/or additive.

37. A method for the treatment of a pathological condition in a subject comprising administering to the subject a therapeutically effective amount of a targeting complex or a pharmaceutical composition comprising the same, said complex comprises:
a. at least one target recognition component comprising a molecule which specifically binds to said pathologic agent or any functional fragment of said agent;
b. an active component comprising an immuno-stimulatory agent; and
c. optionally, a connecting component that associates said at least one targeting component and said at least one active component, wherein the independent biological functions of said targeting component and said active component are retained.

38. The method according to claim 37, wherein said composition is as defined in any one of claims 21 to 23, 25 to 29 and 31 to 33.

39. The method according to claim 37, wherein said complex is as defined by any one of claims 1 to 19.

40. The method according to claim 37, wherein said subject affected by the pathogenic agent is any one of human, domestic mammal, domestic bird, domestic fish, pet and exotic fish.

41. The method according to claim 40, wherein said pathological disorder is any one of immune-related disorders, viral, fungal or bacterial infections and malignant disorders.

42. The method according to claim 41, wherein said pathological disorder is a bacterial infection.

43. A method for the treatment of mastitis in bovine comprising administering to said bovine in need a therapeutically effective amount of a targeting complex capable of inducing specific phagocytosis targeted to at least one target mastitis pathogenic bacteria in said infected bovine, or of a pharmaceutical composition comprising the same, said complex comprises:
a. at least one target recognition component comprising a molecule which specifically binds said mastitis pathogenic bacteria or any functional fragment of said bacteria;
b. an active component comprising phagocytosis inducing agent; and
c. optionally, a connecting component that associates said at least one targeting component and said at least one active component, wherein the independent biological functions of said targeting component and said active component are retained. WO 03/076471 PCT/ILO3/00196

44. The method according to claim 43, wherein said complex is as defined in any one of claims 17 to 19.

45. A method for the treatment of ulcerative disease in fish comprising administering to said fish in need a therapeutically effective amount of a targeting complex capable of inducing specific phagocytosis targeted to at least one target Aeroinonas salinonicida pathogenic bacteria in said infected fish according to claim 20, or of a pharmaceutical composition comprising the same.

46. Use of a multi-functional targeting complex according to any one of claims 1 to 19, in the preparation of a pharmaceutical composition for the treatment of a pathological disorder in a subject in need.

47. Use according to claim 46, wherein said pathological disorder is any one of immune-related disorders, viral, fungal or bacterial infections and malignant disorders.

48. Use according to claim 47, wherein said pathologic disorder is a bacterial infection.

49. Use of a multi-functional targeting complex, according to any one of claims 17 to 19, in the preparation of a pharmaceutical composition for the treatment of mastitis in bovine.

50. Use of a multi-functional targeting complex, according to claim 20, in the preparation of a pharmaceutical composition for the treatment of ulcerative disease in fish.
